SEQUENCE LISTING (4 sequences)

SEQ ID NO: 1 - DNA, 41 bp, Artificial Sequence
  synthetic primer
gcagtccatggagaaaagcgtcggagattgtacttcaaccc

SEQ ID NO: 2 - DNA, 41 bp, Artificial Sequence
  synthetic primer
tagactaagatctgtgctttgtgattctttcaagtacttgg

SEQ ID NO: 3 - DNA, 34 bp, Artificial Sequence
  synthetic primer
acgtccatggcaaaaccccatgagattgtgctag

SEQ ID NO: 4 - DNA, 31 bp, Artificial Sequence
  synthetic primer
cagtagatctgtgcttagagtacttctggag

(19) United States
(12) Patent Application Publication
(10) Pub. No.: US 2005/0060767 A1
(43) Pub. Date: Mar. 17, 2005

(54) DNA shuffling to produce herbicide selective crops

(76) Inventors: Venkiteswaran Subramanian, San Diego, CA (US); Willem P.C. Stemmer, Los Gatos, CA (US); Linda A. Castle, Mountain View, CA (US); Umesh S. Muchhal, Mountain View, CA (US); Daniel L. Siehl, Menlo Park, CA (US)

Correspondence Address: MAXYGEN, INC.;INTELLECTUAL PROPERTY DEPARTMENT, 515 GALVESTON DRIVE, RED WOOD CITY, CA, 94063, US

(21) Appl. No.: 10/627,449

(22) Filed: Jul. 25, 2003

Related U.S. Application Data
(63) Continuation of application No. 09/373,333, filed on Aug. 12, 1999, now abandoned.
(60) Provisional application No. 60/112,746, filed on Dec. 17, 1998.
(60) Provisional application No. 60/111,146, filed on Dec. 7, 1998.
(60) Provisional application No. 60/096,288, filed on Aug. 12, 1998.
(60) Provisional application No. 60/096,271, filed on Aug. 12, 1998.
(60) Provisional application No. 60/130,810, filed on Apr. 23, 1999.

(51) Int. Cl. A01H 1/00; C12N 15/82

(52) U.S. Cl. 800/278; 435/468

(57) ABSTRACT

Methods of shuffling DNA to obtain recombinant herbicide tolerance nucleic acids encoding proteins having new or improved herbicide tolerance activities, libraries of shuffled herbicide tolerance nucleic acids, transgenic plants and DNA shuffling mixtures are provided.

### CROSS REFERENCE TO RELATED APPLICATIONS

This application claims the benefit under 35 U.S.C. §119(e) of U.S. Provisional Application No. 60/112,746 filed Dec. 17, 1998, U.S. Provisional Application No. 60/111,146 filed Dec. 7, 1998, and U.S. Provisional Application No. 60/096,288 filed Aug. 12, 1998, all of which are incorporated herein by reference, and additionally includes subject matter related to U.S. Provisional Application No. 60/096,271 filed Aug. 12, 1998, U.S. Provisional Application No. 60/130,810 filed Apr. 23, 1999, and a U.S. Patent Application entitled “DNA SHUFFLING OF MONOOXYGENASE GENES FOR PRODUCTION OF INDUSTRIAL CHEMICALS” (Attorney Docket No. 18097-025820US), filed on date even herewith.

### FIELD OF THE INVENTION

This invention pertains to the shuffling of nucleic acids to achieve or enhance herbicide tolerance.

### BACKGROUND OF THE INVENTION

Herbicides are universally applied in modem agriculture to control weed growth in crop fields. The strategy for application of herbicides to kill weeds without harming crop plants is dependent on selective tolerance to a given herbicide by certain crop plants. In other words, crop plants survive application of the herbicide without significant ill effect, while weed plants do not.

“Crop selectivity” is defined as the ability of crops to survive herbicide treatments without visible injury (or at least with minimal injury) as compared to control of a weed target by the herbicide. The fact that herbicides are used in crops implies that they are safe (selective) to crops, while providing total or at least acceptable control to economically important weeds.

Crop selectivity is determined by the inherent ability of different crops to metabolize specific herbicides more rapidly than the weeds targeted by an herbicide. See, Owen (1989) “Metabolism of Herbicides—Detoxification as the Basis of Selectivity” In: Herbicides and Plant Metabolism (Dodge A D, ed), pp 171-198, Cambridge University Press, Cambridge, UK (“Owen, 1989”), and Owen and deBoer (1995) “Plant Metabolism and the Design of New Selective Herbicides” In: Eighth International Congress of Pesticide Chemistry (Ragsdale N N, Kearney P C and Plimmer J R, eds), pp 257-268, American Chemical Society, Washington, DC. (“Owen, 1995”).

Because there are many different crop plants grown in agriculture, a given herbicide is well tolerated by some crop plants, but not by others. Where the genes conferring tolerance in one crop species are known, they can often be transferred into a second crop species to make the second species resistant as well. In general, genes which confer tolerance can be engineered into plants, regardless of the source of the gene.

For example, crop selectivity to specific herbicides can be conferred by engineering genes into crops which encode appropriate herbicide metabolizing enzymes from other organisms, such as microbes. See, Padgette et al. (1996) “New weed control opportunities: Development of soybeans with a Round UP Ready™ gene” In: Herbicide-Resistant Crops (Duke, ed.), pp 53-84, CRC Lewis Publishers, Boca Raton (“Padgette, 1996”); and Vasil (1996) “Phosphinothricin-resistant crops” In: Herbicide-Resistant Crops (Duke, ed.), pp 85-91, CRC Lewis Publishers, Boca Raton) (“Vasil, 1996”).

Indeed, transgenic plants have been engineered to express a variety of herbicide tolerance/metabolizing genes, from a variety of organisms. For example, acetohydroxy acid synthase, which has been found to make plants which express this enzyme resistant to multiple types of herbicides, has been cloned into a variety of plants (see, e.g., Hattori, J., et al. (1995) Mol. Gen. Genet. 246(4):419). Other genes that confer tolerance to herbicides include: a gene encoding a chimeric protein of rat cytochrome P4507A1 and yeast NADPH-cytochrome P450 oxidoreductase (Shiota, et al. (1994) Plant Physiol. 106(1)17), genes for glutathione reductase and superoxide dismutase (Aono, et al. (1995) Plant Cell Physiol. 36(8):1687, and genes for various phosphotransferases (Datta, et al. (1992) Plant Mol. Biol. 20(4):619.

Similarly, crop selectivity can be conferred by altering the gene coding for an herbicide target site so that the altered protein is no longer inhibited by the herbicide (Padgette, 1996). Several such crops have been engineered with specific microbial enzymes to confer selectivity to specific herbicides (Vasil, 1996).

A large number of genes which have properties potentially useful for conferring herbicide tolerance are known. Two major classes of enzymes involved in conferring natural crop selectivity to herbicides are (a) monooxygenases such as cytochrome P450 monooxygenases (P450s) and (b) glutathione sulfur-transferases (GSTs) and homoglutathione sulfur-transferases (HGSTs) (Owen 1989, 1995). For example, several hundred cytochrome P450 genes, which encode enzymes that mediate a variety of chemical processes in the cell, have been cloned or otherwise characterized. For an introduction to cytochrome P450, see, Ortiz de Montellano (ed.) (1995) Cytochrome P450 Structure Mechanism and Biochemistry, Second Edition Plenum Press (New York and London) (“Ortiz de Montellano, 1995”) and the references cited therein. Indeed, the large number of readily available genes which potentially encode herbicide tolerance presents a considerable task for screening the genes for herbicide tolerance.

Similarly, there are a wide variety of compounds which are known that kill plants, making them potential herbicides, but for which tolerance factors have not been identified. Even if the large number of known potential herbicide tolerance genes are screened for an ability to metabolize such a compound, there is no assurance that any gene will be identified that provides tolerance to the herbicide. It has been estimated that 30,000 or more compounds with herbicidal activity are typically screened to identify a single crop-selective herbicide. See, e.g., Subramanian et al. (1997) “Engineering dicamba selectivity in crops: A search for appropriate degradative enzyme(s).” J Ind. Microbiol. 19:344-349 (“Subramanian, 1997”) and the references cited therein.

Finally, potential herbicide tolerance genes did not, typically, evolve specifically for the task of herbicide metabolism. Xenobiotic cytochrome P450 genes, for example, are present in organisms as diverse as yeast, bacteria, plants, vertebrates and invertebrates, serving as general cellular enzymes capable of a very wide variety of reactions, including hydroxylations, epoxidations, N-, S-, and O-dealkylations, N-oxidations, sulfoxidations, dehalogenations, and a variety of other reactions. In many organisms, it is clear that there are multiple isoforms of P450 present in cells of the organism, with different isoforms having different substrate specificities. Thus, the fact that some forms of P450 are differentially better at herbicide metabolism than other P450s (i.e., those naturally found in weeds) is often simply serendipitous. While it is often theoretically possible to determine what specific structural features make a particular form of a P450 (or, other protein encoded by a potential herbicide tolerance gene) able to confer herbicide tolerance, and thereby provide insight into how the gene can be modified to improve tolerance, the effort involved in this task can be quite considerable.

Surprisingly, the present invention provides a strategy for solving each of the problems outlined above, as well as providing a variety of other features which will be apparent upon review.

### SUMMARY OF THE INVENTION

In the present invention, DNA shuffling techniques are used to generate new or improved herbicide tolerance genes. These herbicide tolerance genes are used to confer herbicide tolerance in plants such as commercial crops. These new or improved genes have surprisingly superior properties as compared to naturally occurring genes.

In the methods for obtaining herbicide tolerance genes, a plurality of variant forms derived from a parental nucleic acid, or from more than one parental nucleic acid, are recombined. The plurality of variant forms include segments derived from the parental nucleic acid. The parental nucleic acid encodes a herbicide tolerance activity, or, can be shuffled to encode a herbicide tolerance activity and as such is a candidate for DNA shuffling to develop or evolve a herbicide tolerance activity. The plurality of variant forms of the parental nucleic acid differ from each other in at least one (and typically two or more) nucleotides and, upon recombination, provide a library of recombinant nucleic acids. The library can be an in vitro set of molecules, or present in cells, phage or the like. The library is screened to identify at least one recombinant herbicide tolerance nucleic acid that encodes an activity which confers herbicide tolerance to a cell. The recombinant herbicide tolerance nucleic acid can encode a distinct or improved herbicide tolerance activity compared to the activity encoded by the parental nucleic acid or nucleic acids.

The parental nucleic acids to be shuffled can be from any of a variety of sources, including synthetic or cloned DNAs. The parental nucleic acids can encode an herbicide tolerance activity. Alternatively the parental nucleic acids do not encode an herbicide tolerance activity but produce a nucleic acid encoding an herbicide tolerance activity upon recombining variant forms of the parental nucleic acid. Alternatively, the parental nucleic acid encodes a polypeptide which is functionally and/or structurally related to a native herbicide target protein, and can produce a nucleic acid encoding an activity which can substitute for that of the native herbicide target protein upon recombining variant forms of the parental nucleic acid.

Exemplar parental nucleic acids for recombination include genes encoding P450 monooxygenases, glutathione sulfur transferases, homoglutathione sulfur transferases, glyphosate oxidases, phosphinothricin acetyl transferases, dichlorophenoxyacetate monooxygenases, acetolactate synthases, 5-enol pyruvylshikimate-3-phosphate synthases, and UDP-N-acetylglucosamine enolpyruvyltransferases. For example, P450 monooxygenase genes from corn and wheat encode activities which confer tolerance to the herbicide dicamba, making these genes suitable targets for shuffling. Similarly, glutathione sulfur transferase genes from maize, homoglutathione sulfur transferase genes from soybean, glyphosate oxidase genes from bacteria, phosphinothricin acetyl transferase genes from bacteria, dichlorophenoxyacetate monooxygenase genes from bacteria, acetolactate synthase genes from plants, protoporphyrinogen oxidase genes from plants and algae, 5-enolpyruvylshikimate-3-phosphate synthase genes from plants and bacteria, and UDP-N-acetylglucosamine enolpyruvyltransferase genes from bacteria, are all preferred sources for DNA to be shuffled. Allelic and interspecific variants of a parental nucleic acid can be used in these shuffling techniques. Variant forms produced by chemically synthesizing a plurality of nucleic acids homologous to the parental nucleic acid, or produced by error-prone transcription of the parental nucleic acid, or produced by replication of the parental nucleic acid in a mutator cell strain, can also be used in these shuffling techniques.

A variety of screening methods can be used to screen the library of recombinant nucleic acids produced by shuffling, depending on the herbicide against which the library is selected. By way of example, the library to be screened can be present in a population of cells. The library is screened by growing the cells in or on a medium comprising the herbicide and selecting for a detected physical difference between the herbicide and a modified form of the herbicide in the cell. Exemplary herbicides include dicamba, glyphosate, bisphosphonates, sulfentrazones, imidazolinones, sulfonylureas, and triazolopyrimidines. For example, oxidation of the herbicide can be monitored, preferably by spectroscopic methods, thereby providing a measure of how effective the activities encoded by the library are at metabolizing the herbicide. Similarly, glutathione conjugation to an herbicide or herbicide metabolite, or homoglutathione conjugation to an herbicide or herbicide metabolite can also be selected for, based upon a difference in the physical properties of an herbicide before and after conjugation. Alternatively, the library is screened by growing the cells in or on a medium comprising the herbicide and selecting for enhanced growth of the cells in the presence of the herbicide. Enhanced growth of the cell could require the presence of the activity encoded by the recombinant herbicide tolerance nucleic acid. In one variation, the encoded activity is a herbicide metabolic activity, and the cells require the metabolic product of the herbicide for growth. Finally, herbicide tolerance activity to more than one herbicide can simultaneously be screened or selected for in a library, i.e., with the goal of identifying a recombinant herbicide tolerance nucleic acid (or nucleic acids) that encode tolerance activities to more than one herbicide.

Iterative screening and selection for herbicide tolerance is also a feature of the invention. In these methods, a nucleic acid identified as conferring an herbicide tolerance activity to a cell can be further shuffled, either with parental nucleic acids, or with other nucleic acids (e.g., variant forms of the parental nucleic acid) to produce a second shuffled library. The second shuffled library is then screened for one or more herbicide tolerance activity, which can be a tolerance activity to the same herbicide as in the first round of screening, or to a different herbicide. This process can be iteratively repeated as many times as desired, until a recombinant herbicide tolerance nucleic acid with optimized properties is obtained. If desired, recombinant herbicide tolerance nucleic acids identified by any of the methods described herein can be cloned and, optionally, expressed. For example, the nucleic acid can be transduced into a plant to confer a herbicide tolerance activity to the plant. If desired, herbicide tolerance activity conferred to the plant can be tested, e.g., by field testing the herbicide tolerance of the plant.

The invention also provides methods of increasing herbicide tolerance in a plant cell by whole genome shuffling. In these methods, a plurality of genomic nucleic acids are shuffled in the plant cell. The recombined plant cells are screened for one or more herbicide tolerance activities, such as tolerance to herbicides including, for example, dicamba, glyphosate, bisphosphonate, sulfentrazone, an imidazolinone, a sulfonylurea, a triazolopyrimidine, a diphenyl ether, a chloroacetamide, hydantocidin, and the like. The genomic nucleic acids can be from a species or strain different from the plant cell in which herbicide tolerance is desired. Similarly, the shuffling reaction can be performed in cells using genomic DNA from the same or different species or strains. In any case, the plant cell, or a descendent cell thereof, is typically regenerated into a plant which has the desired herbicide tolerance activity.

The distinct or improved herbicide tolerance activity encoded by a herbicide tolerance nucleic acid of the present invention includes one or more of a variety of activities: an increase in ability to metabolize (ie., chemically modify or degrade) the herbicide, an increase in the range of herbicides to which the activity confers tolerance (e.g., tolerance activity to a broader range of herbicides than the activity encoded by the parental nucleic acid), an increase in expression level compared to that of a polypeptide encoded by the parental nucleic acid; a decrease in susceptibility to inhibition by the herbicide compared to that of an activity encoded by the parental nucleic acid; a decrease in susceptibility to protease cleavage compared to that of a polypeptide encoded by the parental nucleic acid; a decrease in susceptibility to high or low pH levels compared to that of a polypeptide encoded by the parental nucleic.acid; a decrease in susceptibility to high or low temperatures compared to that of a polypeptide encoded by the parental nucleic acid; and a decrease in toxicity to a host plant compared to that of a polypeptide encoded by the selected nucleic acid.

One feature of the invention is production of libraries and shuffling mixtures for use in the methods as set forth above. For example, a phage display library comprising shuffled forms of a nucleic acid is provided. Similarly, a shuffling mixture comprising at least three homologous DNAs, each of which is derived from a parental nucleic acid encoding a polypeptide or fragment thereof is provided. These parental nucleic acids can encode polypeptides including, for example, P450 monooxygenase polypeptides, glutathione sulfur transferase polypeptides, homoglutathione sulfur transferase polypeptides, glyphosate oxidase polypeptides, phosphinothricin acetyl transferase polypeptides, dichlorophenoxyacetate monooxygenase polypeptides, acetolactate synthase polypeptides, protoporphyrinogen oxidase polypeptides, 5-enolpyruvylshikimate-3-phosphate synthase polypeptides, UDP-N-acetylglucosamine enolpyruvyltransferase polypeptides, or variant forms thereof.

Recombinant herbicide tolerance nucleic acids identified by screening and selection of the libraries prepared by the methods above are also a feature of the invention.

The invention further provides methods of evaluating long-term efficacy of a herbicide with respect to evolved variants of a plant. These methods entail delivering a library of DNA fragments into a plurality of plant cells, at least some of which undergo recombination with segments in the genome of the cells to produce modified plant cells. Modified plant cells are propagated in a media containing the herbicide, and surviving cells are recovered. DNA from surviving cells is recombined with a further library of DNA fragments at least some of which undergo recombination with cognate segments in the DNA from the surviving cells to produce further modified plant cells. Further modified plant cells are propagated in media containing the herbicide, and further surviving plant cells are collected. The recombination and selection steps are repeated as needed, until a further surviving plant cell has acquired a predetermined degree of resistance to the herbicide. The degree of resistance acquired and the number of repetitions needed to acquire it provide a measure of the efficacy of the herbicide in killing evolved variants of the plant. The information from this analysis is of value in comparing the relative merits of different herbicides and, in particular, in evaluating the long-term efficacy of such herbicides upon repeated administration to weeds.

### BRIEF DESCRIPTION OF THE FIGURE

FIG. 1 shows a strategy for family shuffling of bacterial EPSPS genes to generate libraries that can be screened and selected for recombinant herbicide tolerance nucleic acids encoding glyphosate tolerance activity.

### DEFINITIONS

Unless clearly indicated to the contrary, the following definitions supplement definitions of terms known in the art.

A “recombinant” nucleic acid is a nucleic acid produced by recombination between two or more nucleic acids, or any nucleic acid made by an in vitro or artificial process. The term “recombinant” when used with reference to a cell indicates that the cell comprises (and optionally replicates) a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell where the genes are modified and re-introduced into the cell by artificial means. The term also encompasses cells that contain a nucleic acid endogenous to the cell that has been artificially modified without removing the nucleic acid from the cell; such modifications include those obtained by gene replacement, site-specific mutation, and related techniques.

A “recombinant herbicide tolerance nucleic acid” is a recombinant nucleic acid encoding a protein having an activity which confers herbicide tolerance to a cell when the nucleic acid is expressed in the cell.

A “nucleic acid encoding an activity” is synonymous with a “nucleic acid encoding a protein having an activity”. Likewise, an “activity encoded by a nucleic acid”is synonymous with an “activity of a protein encoded by a nucleic acid”

An “activity” of a protein (or, an “activity” encoded by a nucleic acid) can include a catalytic (i.e., enzymatic) activity, an inherent physical property of the encoded protein (such as susceptibility to protease cleavage, susceptibility to denaturants, ability to polymerize or depolymerize), or both.

“Herbicide tolerance” is the ability of a cell or plant to survive, grow, and/or reproduce, in the presence of an herbicide.

A “herbicide tolerance activity” or, an “activity which confers herbicide tolerance”, is an activity which, when present in a cell or plant, allows the cell or plant to survive, grow, and/or reproduce, in the presence of an herbicide.

An “herbicide” is a chemical or compound that kills one or more plant, typically a weed plant. Herbicides are normally “selective” for one or more crop plant, i.e., they do not significantly damage the crop, while simultaneously controlling weed growth.

“Herbicide metabolism” refers to modification (by, e.g., oxidation, reduction, acetylation, conjugation, etc.) or degradation of a herbicide, by the action of one or more enzymes, to yield a product which is not toxic to the cell or plant.

A “plurality of variant forms” of a nucleic acid refers to a plurality of homologs of the nucleic acid. The homologs can be from naturally occurring homologs (e.g., two or more homologous genes) or by artificial synthesis of one or more nucleic acids having related sequences, or by modification of one or more nucleic acid to produce related nucleic acids. Nucleic acids are homologous when they are derived, naturally or artificially, from a common ancestor sequence. During natural evolution, this occurs when two or more descendent sequences diverge from a parent sequence over time, i.e., due to mutation and natural selection. Under artificial conditions, divergence occurs, e.g., in one of two ways. First, a given sequence can be artificially recombined with another sequence, as occurs, e.g., during typical cloning, to produce a descendent nucleic acid. Alternatively, a nucleic acid can be synthesized de novo, by synthesizing a nucleic acid which varies in sequence from a given parental nucleic acid sequence.

When there is no explicit knowledge about the ancestry of two nucleic acids, homology is typically inferred by sequence comparison between two sequences. Where two nucleic acid sequences show sequence similarity it is inferred that the two nucleic acids share a common ancestor. The precise level of sequence similarity required to establish homology varies in the art depending on a variety of factors. For purposes of this disclosure, two sequences are considered homologous where they share sufficient sequence identity to allow recombination to occur between two nucleic acid molecules. Typically, nucleic acids require regions of close similarity spaced roughly the same distance apart to permit recombination to occur. Typically regions of at least about 60% sequence identity or higher are optimal for recombination.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides, refers to two or more sequences or subsequences that have at least about 60%, preferably 80%, most preferably 90-95% nucleotide or amino acid residue identity, when compared and aligned for maximum correspondence, as measured using one of the following sequence comparison algorithms or by visual inspection. Such “substantially identical” sequences are typically considered to be homologous. Preferably, the “substantial identity” exists over a region of the sequences that is at least about 50 residues in length, more preferably over a region of at least about 100 residues, and most preferably the sequences are substantially identical over at least about 150 residues, or over the full length of the two sequences to be compared.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical/homologous is that the two molecules hybridize to each other under stringent conditions. The phrase “hybridizing specifically to,” refers to the binding, duplexing, or hybridizing of a molecule only to a particular nucleotide sequence under stringent conditions, including when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization experiments such as Southern and northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays,” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but not to unrelated sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCi at 72° C. for about 15 minutes. An example of stringent wash conditions is a 0.2×SSC wash at 65° C. for 15 minutes (see, Sambrook, infra., for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

A further indication that two nucleic acid sequences or polypeptides are substantially identical/homologous is that the polypeptide encoded by the first nucleic acid is immunologically cross reactive with, or specifically binds to, the polypeptide encoded by the second nucleic acid. Thus, a polypeptide is typically substantially identical to a second polypeptide, for example, where the two peptides differ only by conservative substitutions.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton (1984) Proteins, W. H. Freeman and Company. In addition, individual substitutions, deletions or additions which alter, add or delete a single amino acid or a small percentage of amino acids in an encoded sequence are also “conservatively modified variations.” Sequences that differ by conservative variations are generally homologous.

A “subsequence” refers to a sequence of nucleic acids or amino acids that comprise a part of a longer sequence of nucleic acids or amino acids (e.g., polypeptide) respectively. A subsequence of a particular nucleic acid or polypeptide may also be referred to as a “fragment” or a “segment” of the nucleic acid or polypeptide.

The term “gene” is used broadly to refer to any segment of DNA associated with expression of a given RNA or protein. Thus, genes include sequences encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have desired parameters.

The term “isolated”, when applied to a nucleic acid or protein, denotes that the nucleic acid or protein is essentially free of other cellular components with which it is associated in the natural state.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260: 2605-2608; Cassol et al. (1992); Rossolini et al. (1994) Mol. Cell. Probes 8: 91-98). The term nucleic acid is generic to the terms “gene”, “DNA,” “cDNA”, “oligonucleotide,” “RNA,” “mRNA,” and the like.

“Nucleic acid derived from a gene” refers to a nucleic acid for whose synthesis the gene, or a subsequence thereof, has ultimately served as a template. Thus, an MRNA, a cDNA reverse transcribed from an mRNA, an RNA transcribed from that cDNA, a DNA amplified from the cDNA, an RNA transcribed from the amplified DNA, etc., are all derived from the gene and detection of such derived products is indicative of the presence and/or abundance of the original gene and/or gene transcript in a sample.

A nucleic acid is “operably linked” when it is placed into a functional relationship with another nucleic acid sequence. For instance, a promoter or enhancer is operably linked to a coding sequence if it increases the transcription of the coding sequence.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette may also include a nucleic acid that encodes a signal or localization peptide which facilitates translocation of the expressed polypeptide to an intracelluar organelle or compartment (e.g., chloroplast) or for secretion across a membrane. Transcription termination signals, enhancers, and other nucleic acid sequences that influence gene expression, can also be included in an expression cassette.

### DETAILED DISCUSSION OF THE INVENTION

Introduction

Discovery of crop-selective herbicides is a long and arduous process. See, e.g., Parry (1989) “Herbicide use and inventions” In: Herbicides and Plant Metabolism (Dodge AD, ed), pp 1-36, Cambridge University Press, Cambridge, UK. Thousands of chemicals are initially screened for activity on select weeds. Those compounds showing activity are considered as leads for further follow-up synthesis and optimization of activity. During this process, crop selectivity is achieved by incorporating various metabolic handles in the basic toxophore with the hope that one or more crops will rapidly metabolize a few of these analogs. Thus, incorporating crop selectivity in a basic toxophore is a trial and error synthesis process, although the knowledge of the natural metabolic machinery of different crops has been useful (id). It is estimated that discovery of one crop-selective herbicide involves screening more than 30000 compounds (id).

Recent developments in the area of plant biotechnology, notably the ability to stably integrate foreign genes into crops, have opened up an alternative approach to achieving crop selectivity to herbicides. See, e.g., Subramanian (1997), supra. In the last 10 years, several crops have been genetically engineered or selected in tissue culture, to be selective to herbicides (id). For example, glyphosate-selective soybeans were genetically engineered by incorporating a gene that codes for a less sensitive form of 5-enolpyruvyl shikimate-3-phosphate synthase (EPSP synthase). The herbicidal activity of glyphosate is due to inhibition of the wild type EPSP synthase (Padgette, 1996). Similarly, glufosinate selectivity was engineered into maize and other crops by incorporating a bacterial gene that codes for an acetyl transferase (Vasil, 1996). This results in rapid metabolism of the herbicide in the transgenic crops, conferring crop selectivity.

In general, biotechnological approaches to conferring crop selectivity to herbicides involves either: (a) altering the gene that codes for the target site in order to make it less sensitive to a particular herbicide (as in the case with certain glyphosate-selective crops), or (b) engineering into crops, a gene that codes for an enzyme capable of rapid metabolism of a particular herbicide (as is the case of glufosinate-selective crops, see, Subramanian, 1997). Traditionally, such enzymes are discovered either by extensive screening of microorganisms (Padgette, 1996; Subramanian, 1997; and Dyer (1996) “Techniques for producing herbicide-resistant crops” In: Herbicide-Resistant Crops (Duke S O, ed.), pp 85-91, CRC Lewis Publishers, Boca Raton (“Dyer, 1996”)) or by mutagenesis followed by rigorous selection (Padgette, 1996; Dyer, 1996). In spite of this rigorous scheme, the selected enzymes may not have the ideal properties to confer crop selectivity or to function effectively in transgenic crops (Padgette, 1996).

The present invention overcomes these difficulties by applying DNA shuffling to obtain recombinant herbicide tolerance nucleic acids encoding proteins that exhibit one or more distinct or improved herbicide tolerance activities over those encoded by the parental nucleic acids. The herbicide tolerance nucleic acids are used to confer much higher margins of crop selectivity and safety to different herbicides for better weed control. A number of applications are given below by way of example.

In one general strategy, DNA shuffling is applied to genes or gene families that encode proteins that metabolize (i.e., modify or degrade) the herbicides into inactive (or less active) products. Such genes include those encoding P450 monooxygenase, glutathione sulfur transferase, homoglutathione sulfur transferase, glyphosate oxidase, phosphinothricin acetyl transferase, and dichlorophenoxyacetate monooxygenase. Such genes are optimized by DNA shuffling in order to enhance the rate of metabolism of specific herbicides, optionally without altering other properties, such as stability, or affinity for natural substrates, cofactors, effectors, etc. In another general strategy, DNA shuffling is applied to genes or gene families that encode the protein targets of particular herbicides (ie. “herbicide target proteins”), such as acetolactate synthase, protoporphyrinogen oxidase, and 5-enolpyruvylshikimate-3-phosphate synthase. Such genes are optimized by DNA shuffling in order to reduce the inhibitory activity of specific herbicides on their target proteins, optionally without altering other target protein properties, such as stability, affinity for natural substrates, cofactors, effectors, etc. In another general strategy, DNA shuffling is applied to genes or gene families to acquire new activities which mimic those of native plant herbicide target proteins. The candidate parent genes for shuffling encode proteins having functional and/or structural similarities to the native target protein, and lack, or have reduced, inhibitory activity of specific herbicides compared to the native target protein. Such genes are optimized by DNA shuffling, optionally together with nucleic acids derived from target protein genes, to generate recombinant herbicide tolerance nucleic acids that encode proteins which can functionally substitute for the native herbicide-sensitive target protein.

Methods for modifying a nucleic acid for the acquisition of, or an improvement in, an activity useful in conferring upon plants tolerance to herbicides, are provided, and include, but are not limited to, methods for modifying P450 monooxygenases, glutathione sulfur transferases, homoglutathione sulfur transferases, glyphosate oxidases, phosphinothricin acetyl transferases, dichlorophenoxyacetate monooxygenases, acetolactate synthases, protoporphyrinogen oxidases, 5-enolpyruvylshikimate-3-phosphate synthases, and UDP-N-acetylglucosamine enolpyruvyltransferases. The methods involve using DNA shuffling to obtain recombinant herbicide tolerance genes that, when present in or on a plant, confer herbicide tolerance to the plant.

The invention provides significant advantages over previously used methods for optimization of herbicide tolerance genes. For example, DNA shuffling can result in optimization of a desirable property even in the absence of a detailed understanding of the mechanism by which the particular property is mediated. In addition, entirely new properties can be obtained upon shuffling of DNAs, i.e., shuffled DNAs can encode polypeptides or RNAs with properties entirely absent in the parental DNAs which are shuffled.

Sequence recombination can be achieved in many different formats and permutations of formats, as described in further detail below. These formats share some common principles.

The substrates for modification, or “forced evolution,” vary in different applications, as does the property sought to be acquired or improved. Examples of candidate substrates for acquisition of a property or improvement in a property include genes that encode proteins which have enzymatic or other activities useful in conferring herbicide tolerance.

The methods use at least two variant forms of a starting substrate. The variant forms of candidate substrates can have substantial sequence or secondary structural similarity with each other, but they should also differ in at least one and preferably at least two positions. The initial diversity between forms can be the result of natural variation, e.g., the different variant forms (homologs) are obtained from different individuals or strains of an organism (including geographic variants) or constitute related sequences from the same organism (e.g., allelic variations), or constitute homologs from different organisms (interspecific variants). Alternatively, initial diversity can be induced, e.g., the variant forms can be generated by error-prone transcription (such as an error-prone PCR or use of a polymerase which lacks proof-reading activity; e.g., Liao (1990) Gene 88:107-111) of the first form of the starting substrate, or, by replication of the first form in a mutator strain (mutator host cells are discussed in further detail below, and are generally well known), or by synthesizing a nucleic acid which varies in sequence from that of the first form. The initial diversity between substrates is greatly augmented in subsequent steps of recombination for library generation.

A mutator strain can include any mutants in any organism impaired in the functions of mismatch repair. These include mutant gene products of mutS, mutT, mutH, mutL, ovrD, dcm, vsr, umuC, umuD, sbcB, recj, etc. The impairment is achieved by genetic mutation, allelic replacement, selective inhibition by an added reagent such as a small compound or an expressed antisense RNA, or other techniques. Impairment can be of the genes noted, or of homologous genes in any organism.

The activities or other characteristics that can be acquired or improved vary widely, and, of course depend on the choice of substrate. For example, for herbicide tolerance genes, activities that one can improve include, but are not limited to, increased range of herbicides against which a particular tolerance gene is effective, increased metabolic activity towards an herbicide, increased expression of the tolerance gene, reduced inhibition of activity by the herbicide, decreased susceptibility to protease degradation (or other natural protein or RNA degradative processes), increased activity ranges for conditions such as heat, cold, low or high pH, and reduced toxicity to the host plant.

At least two variant forms of a nucleic acid which can confer herbicide tolerance activity, or which can potentially confer herbicide tolerance activity, are recombined to produce a library of recombinant nucleic acids. The library is then screened to identify at least one recombinant herbicide tolerance gene that is optimized for the particular activity or activities of interest.

Often, improvements are achieved after one round of recombination and screening. However, recursive sequence recombination can be employed to achieve still further improvements in a desired herbicide tolerance activity, or to bring about herbicide tolerance activities new (i.e.,“distinct”) from activities encoded by the parental nucleic acid. Recursive sequence recombination entails successive cycles of recombination to generate molecular diversity. That is, one creates a family of nucleic acid molecules showing some sequence identity to each other but differing in the presence of mutations. In any given cycle, recombination can occur in vivo or in vitro, intracellularly or extracellularly. Furthermore, diversity resulting from recombination can be augmented in any cycle by applying prior methods of mutagenesis (e.g., error-prone PCR or cassette mutagenesis) to either the substrates or products for recombination.

A recombination cycle is usually followed by at least one cycle of screening or selection for nucleic acids encoding a desired herbicide tolerance activity. If a recombination cycle is performed in vitro, the products of recombination (i.e., recombinant segments, recombinant libraries, or “libraries of recombinant nucleic acids”) are sometimes introduced into cells before the screening step. Recombinant libraries can also be linked to an appropriate vector or other regulatory sequences before screening. Alternatively, recombinant libraries generated in vitro are sometimes packaged in viruses (e.g., bacteriophage) before screening. If recombination is performed in vivo, recombinant libraries can sometimes be screened in the cells in which recombination occurred. In other applications, recombinant libraries are extracted from the cells, and optionally packaged as viruses, before screening.

The nature of screening or selection depends on what herbicide tolerance activity is to be acquired or the herbicide tolerance activity for which improvement is sought, and many examples are discussed below. It is not usually necessary to understand the molecular basis by which particular products of recombination (recombinant libraries) have acquired new or improved herbicide tolerance activities relative to the starting substrates. For example, an herbicide tolerance gene can have many component sequences each having a different intended role (e.g., coding sequence, regulatory sequences, targeting sequences, stability-conferring sequences, and sequences affecting integration). Each of these component sequences can be varied and recombined simultaneously. Screening/selection can then be performed, for example, for recombinant segments that have increased ability to confer herbicide tolerance upon a plant without the need to attribute such improvement to any of the individual component sequences.

Depending on the particular screening protocol used for a desired property, initial round(s) of screening can sometimes be performed using bacterial cells due to high transfection efficiencies and ease of culture. Photosynthetic cells, such as cyanobacteria and the unicellular alga Chlamydomonas, are particularly useful for screening activities ultimately destined for plants. Later rounds of screening, and other types of screening which are not amenable to screening in bacterial cells, are performed in plant cells to optimize recombinant segments for use in an environment close to that of their intended use. Final rounds of screening can be performed in the precise cell type of intended use (e.g., a cell which is present in a plant), or even in whole plants (e.g., crop-herbicide tests in the field). Transient gene expression systems may be utilized in screening plant cells for expression of herbicide tolerance activities. In some methods, use of a recombinant herbicide tolerance gene can itself be used as a round of screening. That is, recombinant herbicide tolerance genes that are successfully taken up and/or expressed by the intended target cells are recovered from those target cells and used to confer tolerance upon other plants. The recombinant herbicide tolerance genes that are recovered from the first target cells are enriched for genes that have evolved, i.e., have been modified by recursive sequence recombination, toward improved or new activities or characteristics for specific uptake and integration of the gene, effectiveness against the herbicide, stability, and the like.

The screening or selection step identifies a subpopulation of recombinant nucleic acids that have evolved toward acquisition of a new (“distinct”) or improved herbicide tolerance activity useful in conferring herbicide tolerance upon plants. Depending on the screen, the recombinant nucleic acids can be identified as components of cells, components of viruses or in free form. More than one round of screening or selection can be performed after each round of recombination. Alternatively, more than one round of recombination can be performed to increase the diversity of the recombinant nucleic acid library prior to screening or selection.

If further improvement in a herbicide tolerance activity is desired, at least one and usually a collection of recombinant herbicide tolerance nucleic acids surviving a first round of screening/selection are subject to a further round of recombination. These recombinant herbicide tolerance nucleic acids can be recombined with each other or with exogenous nucleic acids derived, e.g., from the original parental nucleic acids or further variants thereof. Again, recombination can proceed in vitro or in vivo. If the previous screening step identifies desired recombinant herbicide tolerance nucleic acids as components of cells, the components can be subjected to further recombination in vivo, or can be subjected to further recombination in vitro, or can be isolated before performing a round of in vitro recombination. Conversely, if the previous screening step identifies desired recombinant herbicide tolerance nucleic acids in naked form or as components of viruses, these nucleic acids can be introduced into cells to perform a round of in vivo recombination. The second round of recombination, irrespective how performed, generates further recombinant nucleic acids which encompass additional diversity than is present in recombinant nucleic acids resulting from previous rounds.

The second round of recombination can be followed by a further round of screening/selection according to the principles discussed above for the first round. The stringency of screening/selection can be increased between rounds. Also, the nature of the screen and the activity being screened for can vary between rounds if improvement in more than one activity is desired or if acquiring more than one new activity is desired. Additional rounds of recombination and screening can then be performed until the recombinant segments have sufficiently evolved to acquire the desired new or improved herbicide tolerance activity.

The practice of this invention involves the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well-known to persons of skill. General texts which describe molecular biological techniques useful herein, including mutagenesis, include Berger and Kimmel, Guide to Molecular Cloning Techniques, Methods in Enzymology (volume 152) Academic Press, Inc., San Diego, Calif. (“Berger”); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1998) (“Ausubel”). Examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA) are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

Oligonucleotides for use as probes, e.g., in in vitro amplification methods, for use as gene probes, or as shuffling targets (e.g., synthetic genes or gene segments) are typically synthesized chemically according to the solid phase phosphoramidite triester, method described by Beaucage and Caruthers (1981), Tetrahedron Letts., 22(20): 1859-1862, e.g., using an automated synthesizer, as described in Needham-VanDevanter et al. (1984) Nucleic Acids Res., 12:6159-6168. Oligonucleotides can also be custom made and ordered from a variety of commercial sources known to persons of skill.

General Strategies for Obtaining Herbicide Tolerance Nucleic Acids

DNA shuffling can be applied to nucleic acids coding for enzymes involved in metabolism (i.e., modification, degradation) of chemicals, to generate a library that can be screened to identify one or more herbicide tolerance nucleic acids that encode improved metabolic activities towards certain herbicides relative to activities encoded by the parental nucleic acids, or that encode herbicide metabolic activities distinct from activities encoded by the parental nucleic acids.

DNA shuffling can also be applied to nucleic acids coding for proteins that are target sites of certain herbicides, such that the improved proteins are desensitized to herbicide but are relatively unchanged with respect to affinity for natural substrates. Herbicide tolerance nucleic acids encoding the improved proteins are then used to confer crop selectivity to one or more herbicides/herbicide families that inhibit the wild type form of the protein.

DNA shuffling can also be applied to nucleic acids coding for proteins having structural and/or functional similarity to herbicide target proteins, yet are relatively insensitive to the herbicide, to evolve herbicide tolerance nucleic acids encoding proteins that mimic the function of the herbicide target protein and lack the herbicide sensitivity of the target protein.

These three general strategies are illustrated in the following examples, which describe acquisition of tolerance to herbicides such as those prone to metabolism via P450 pathways (e.g., dicamba, sulfonylureas, triazolopyrimidines, and the like), enhancement of herbicide metabolism by conjugative pathways (e.g. triazines, thiocarbamates, chloracetamides, sulfonylureas), and desensitation or functional replacement of herbicide target proteins.

DNA Shuffling to Evolve Herbicide Metabolizing Activities

A. Shuffling of P450 Genes

(i) Dicamba Selectivity

Dicamba (2-methoxy-3,6-dichlorobenzoic acid) is a postemergence herbicide which is used for control of broadleaf weeds in corn and wheat fields. Even though corn, wheat, and other grass crops can metabolize dicamba by the action of cytochrome P450 monooxygenases (Subramanian, 1997; Frear DS (1976) in: Herbicides, Kearney P C and Kaufinan D D, eds., pp 541-594, Marcell Dekker, N.Y. (“Frear, 1976”), native metabolism of the herbicide in these crops is not rapid, and not adequate for flexible use of the herbicide for commercial weed control in grass crops. Moreover, dicot crops are extremely sensitive to dicamba. DNA shuffling can be applied to optimize P450 genes in wheat, corn and other grass crops, for rapid metabolism of dicamba to provide higher margins of crop selectivity to the herbicide. An optimized dicamba-metabolizing P450 gene can also be used to confer dicamba-selectivity to dicot crops like soybeans.

Genes coding for dicamba-metabolizing cytochrome P450 monooxygenases can be isolated from cDNA libraries of corn, wheat, or other grasses, by using consensus sequence as primers (Hotze M et al., (1995) FEBS Letters, 374: 345-350, Frey M et al., (1995) Mol. Gen. Genetics, 246:100-109). The isolated genes can be functionally expressed in yeast (Batard Y. (1998) The Plant Journal 14: 111-120) or in E. coli (Anderson J F (1994) Biochemistry 33: 2171-2177) containing P450 reductase. Clones expressing P450 genes are confirmed for activity versus dicamba by, e.g. preparing extracts and assaying for dicamba oxidation activity. The expected product of dicamba oxidation, 5-hydroxydicamba, can be separated from the parent compound, e.g., by HPLC (Subramanian, 1997). Clones containing nucleic acids encoding dicamba oxidation activity may also be identified by growth in a minimal medium containing the herbicide as a sole carbon source. Clones containing P450 encoding dicamba oxidation activity fluoresce due to formation of 5-hydroxydicamba.

P450 genes encoding dicamba oxidation activity can also be isolated by screening a number of cloned cytochrome P450 monooxygenases from various sources for activity versus dicamba. The screen can be conducted by measuring dicamba oxidation activity as described above. The cloned P450s are optionally of microbial, plant, insect or mammalian origin. Genes encoding dicamba metabolizing enzymes may also be isolated by: (a) directly screening microorganisms for growth on dicamba and/or (b) by screening for dicamba metabolizing activity after growth on analogs of dicamba such as chloro or methoxy benzoate (Subramanian, 1997). Method (b) in particular has the potential to discover a wide variety of enzymes capable of metabolizing dicamba.

P450 gene(s) isolated by any of the above methods and encoding dicamba oxidizing activity, can be shuffled by a variety of different approaches to improve activity. In one approach, DNA shuffling can be performed on a single parental gene, as described in more detail below. In another approach, several homologous genes can be utilized in the shuffling reaction. Homologous P450 genes can be identified by comparing the sequences of isolated genes. Homologous P450 sequences, irrespective of the function of the P450, can also be found from GenBank or other sequence repositories. Ortiz de Montellano, 1995, and the references therein provide considerable detail on P450 structure and function. Representative alignments of P450 enzymes can be found in the appendices of Ortiz de Montellano, 1995. An up-to-date list of P450 genes is also found electronically on the World Wide Web at http://dmelson.utmem.edu/cytochromep450.html.

The P450 genes, or fragments thereof, are typically synthesized and shuffled as described in more detail below. Gene shuffling and family shuffling provide two of the most powerful methods available for improving and “migrating” (i.e., gradually changing the type of reaction, substrate specificity or activity to one distinct from that encoded by the parental nucleic acid) the functions of biocatalysts. In gene shuffling, a parental nucleic acid is mutated or otherwise altered to produced variants forms, and then the variant forms are recombined. In family shuffling, homologous sequences, e.g. from different species or chromosomal positions, are recombined.

The shuffled genes can be cloned, e.g., into E. coli containing cytochrome P450 reductase, and those producing high activity on dicamba are identified. First, clones expressing P450 can be examined for dicamba oxidation activity, e.g., in pools of about 10 in order to rapidly screen the initial transformants. Any pools showing significant activity can be deconvoluted (e.g., cloned by limiting dilution) to identify single desirable clones with high activity.

The P450 gene from one or more such clones is optionally subjected to a second round of shuffling in order to further optimize the rate of oxidation of dicamba. E. coli transformants containing the shuffled P450 genes can be grown directly on a medium containing dicamba and those capable of oxidation are identified by fluorescence of the product. The intensity of fluorescence is useful in selecting those clones with high level of activity. Eventually, colonies selected directly from the fluorescence screen are further assayed in crude extract to quantitate dicamba metabolizing activity. Again, the P450 gene from one or more such clones can be subjected to iterative shuffling to further optimize the rate of dicamba oxidation.

Although discussed above for simplicity with reference to P450 monooxygenase gene, it will be appreciated that the same cloning, shuffling, and screening approaches for gene optimization can be applied to other genes to obtain a recombinant herbicide tolerance nucleic acid encoding a distinct or improved metabolizing activity against dicamba. Indeed, as discussed below, whole genome shuffling, which does not require any knowledge about the starting genes to be screened, can be performed using the screening approaches discussed herein. In general, enzymes which have potential activity against dicamba and which are, therefore, suitable for shuffling include known monooxygenases, e.g., those capable of epoxidation such as the monooxygenase from P. oleovorans (May et al. (1973) J. Biol. Chem. 248:725-1730; May et al, J. Am. Chem. Soc. 98:7856-7858). Indeed, the non-heme iron-sulfur monooxygenase system of Pseudomonas oleovorans is among the most well studied system for catalyzing monooxygenase reactions and homologous enzymes have also been identified in several genera including Rhodococcus, Mycobacterium, Pseudomonas and Bacillus.

The recombinant herbicide tolerance nucleic acid optimized for rapid oxidation of dicamba is used to provide higher margins of selectivity in transgenic maize and wheat and enhance the window of application of dicamba to these crops. In addition, the optimized nucleic acid is used to provide dicamba selectivity in dicot crops such as soybean, where this herbicide is not currently used. Methods of transferring genes into essentially any plant are available and discussed in more detail below.

(ii) Other Herbicide Selectivities

As genes of the P450 superfamily encode activities which modify a variety of compounds, DNA shuffling can be applied to a P450 gene or to a family of P450 genes to evolve one or more herbicide tolerance nucleic acids encoding activities for metabolism of other herbicides. P450 genes from a wide variety of sources including microbes, insects, plants and animals can be shuffled to evolve herbicide tolerance nucleic acid(s) capable of rapid metabolism of nonselective herbicides. Such nucleic acids can be used to confer crop selectivity to nonselective herbicides. Several herbicides are known in the art, such as sulfonylureas (Hinz et al. (1995) Weed Science 45: 474-480), and triazolopyrimidines (Owen, 1995), to be metabolized by P450s .

For example, DNA shuffling can be applied to obtain a herbicide tolerance nucleic acid capable of rapid metabolism of a nonselective herbicide, such as, bisphosphonate, sulfentrazone, sulfonylurea, imidazolinone, and the like. All of the cloning, shuffling, screening, selection and optimization procedures described herein can be applied for evolving a parental gene or gene family, such as a P450 gene or gene family, to produce a recombinant nucleic acid encoding metabolizing activity for a given herbicide. The screening can thus be based on differences in the physical properties between the substrate herbicide and its modified product. The recombinant herbicide tolerance nucleic acid encoding an optimized herbicide metabolic activity is used to provide selectivity to different transgenic crops for a given herbicide.

DNA shuffling can also be applied to obtain a broad-specificity herbicide tolerance nucleic acid encoding an activity capable of rapid metabolism of more than one herbicide. All of the screening, cloning, shuffling, selection and optimization procedures described herein can be applied for shuffling, e.g., a P450 gene or gene family to obtain a broad-specificity herbicide tolerance nucleic acid. The screening is typically based on differences in the physical properties between the substrate herbicide(s) and modified product(s). The recombinant herbicide tolerance nucleic acid encoding an activity optimized for rapid metabolism of several herbicides is used to provide selectivity to different transgenic crops for a number of herbicides, which can be used individually, or as mixtures. It will be appreciated that it is more difficult for weed plants to develop tolerance to multiple herbicides simultaneously; thus, crop plants which tolerate simultaneous application of multiple herbicides can be especially valuable.

B. Shuffling of Glutathione- and Homoglutathione Transferase Genes

DNA shuffling can be applied to optimize genes coding for metabolic conjugation enzymes such as glutathione sulfur-transferase (GST) or homoglutathione sulfur-transferase (HGST) from plants (e.g., crops such as maize and soybean), as well as from other sources such as insects, bacteria and animals, for rapid metabolism of herbicides such as triazines, thiocarbamates, chloracetamides, sulfonylureas, or other herbicides which are metabolized or capable of metabolism by GST or HGST. The optimized genes are used to confer enhanced margins of crop selectivity to these herbicides or to confer selectivity to certain crops that were previously sensitive to one of the above herbicides.

Conjugation to glutathione by the action of GST is one of the major mechanisms of detoxification of herbicides in maize (Edwards R. Brighton Crop Protection Conference—Weeds—1995, 823-832). Maize has several isozymes of GST with varying activity towards different compounds, including herbicides. Similarly, soybeans detoxify some herbicides via conjugation to homoglutathione, a glutathione analog (Owen, 1995). This reaction is catalyzed by homoglutathione sulfur-transferase (HGST).

Although GST and HGST catalyze very similar reactions using closely related analogs as conjugating substrates, they do not generally metabolize the same herbicide. Also, maize-selective herbicides known to be detoxified by GST do not show similar margins of selectivity in soybeans. Therefore, in another embodiment, DNA shuffling is applied to GST or HGST nucleic acids, or to a combination of GST and HGST nucleic acids, to evolve a transferase which accepts both glutathione and homoglutathione as substrates. The optimized GST/HGST transferase nucleic acids are used, for example, to produce transgenic corn and soybean that are resistant to the same herbicide.

Genes encoding GST isozymes from maize can be isolated and cloned (Shah D M et al. (1986) Plant Mol. Biology 6: 203-211) by using consensus sequences available for the genes. HGST gene from soybean can be isolated, e.g., using primers derived from the nucleic acid sequence or from back-translation of the protein sequence. Homologs of GST and HGST are also identified from GenBank or other sequence repositories by sequence comparison analysis (for example, by selecting sequences which have a set percent identity, e.g., as described in detail above). Genes can be synthesized (or PCR amplified or cloned from appropriate source materials), shuffled, typically by family shuffling, cloned and introduced into cells such as E. coli. Transformants expressing active GST and HGST can be screened by direct enzyme assays, e.g., in pools of about ten transformants. Assays can be performed either in crude extract or upon rapid purification of the enzyme via, for example, a glutathione affinity column. Substrate herbicide and the conjugated product can be separated by HPLC and quantitated. Alternately, mass spectrometry can be used to track the conjugated product. Pools showing significant activity are deconvoluted to identify the single desirable clone with high activity. The GST/HGST gene from one or more such clones may be subjected to a second round of shuffling to fuirther optimize the reaction rate. If the substrate herbicide inhibits growth of the cells, shuffled genes can be directly selected on the herbicide, since the herbicide conjugates are generally non-toxic. In such a situation, colony size of the transformants would indicate the activity of the shuffled gene product. Activity can also be confirmed by direct quantitative assay using extracts prepared from positive clones. Again, the GST/HGST genes from one or more such clones could be subjected to a iterative shuffling for optimization.

C. Shuffling of Other Metabolic Genes for Herbicide Tolerance

DNA shuffling can be applied to other genes or gene families of plant or non-plant origin to generate libraries that can be screened to identify one or more recombinant herbicide tolerance nucleic acids that encode distinct or improved activities which metabolize (i.e., degrade or modify) a particular herbicide, or a variety of herbicides, to non-phytotoxic products.

The first enzyme involved in the degradation of syringic acid in Clostridium thermoaceticum is active on dicamba, converting it to 3,6-dichlorosalicylic acid (DCSA; el Kasmi A. et al. (1994) Biochemistry 33: 11217-11224). Nucleic acids encoding this enzyme, as well as homologs identified by sequence comparison against e.g., the GenBank database, may be isolated or synthesized by methods described herein or otherwise known to those of skill in the art. The gene can be shuffled, either singly or with homologous sequences. The shuffled genes can be cloned and introduced into cells, such as E. coli, and those producing high activity on dicamba can be identified by methods described above, or by fluorescence-based screening for formation of DCSA. Clones selected with respect to a high rate of activity in a dicamba screen can be further assayed in crude extract to quantitate the activity. Selected genes may be subjected to iterative shuffling to further optimize the rate of dicamba metabolism. Other plant or non-plant genes known or suspected to encode activities which metabolize dicamba (as described in, for example, Subramanian, 1997) or metabolize other herbicides may be isolated and optimized by DNA shuffling to provide herbicide tolerance nucleic acids of the present invention.

The bar gene encodes phosphinothricin acetyl transferase (PAT) which acetylates the herbicide phosphinothricin to a non-toxic product. A gene encoding PAT from Streptomyces hygroscopicus is published in GenBank under accession number XI 7220. Variant forms derived from the published sequence, or segments thereof, may be shuffled in single-gene formats. In addition, homologous sequences can be found by homology-searching the GenBank database against the published sequence; the homologous sequences may be used to prepare additional nucleic acid substrates to be used in family shuffling formats. Clones are screened based on increased rates of acetyl-phosphinothricin formation.

DNA shuffling can also be applied to enhance the activity of an enzyme involved in the metabolism of glyphosate to an inactive product. One such enzyme is the microbial enzyme glyphosate oxidase (GOX; Padgette, 1996). A gene coding for this enzyme is isolated by screening genomic DNA preparations of Achromobacter in a Mpu⁺ E. coli strain with glyphosate as the sole phosphorous source (Padgette, 1996). The selection is based on the fact that growth of this E. coli strain is inhibited by glyphosate. Introduction of the glyphosate oxidase gene restores growth due to the conversion of glyphosate to aminomethylphosphonate, which is readily utilized by the Mpu⁺ strain as carbon and phosphorous source. GOX genes are shuffled and screened in the Mpu⁺ strain in the presence of glyphosate, where larger colony size is indicative of enhanced oxidase activity. This is confirmed by direct measurement of glyphosate metabolism in crude extracts. Shuffled and optimized genes encoding improved glyphosate oxidation activity are used to confer selectivity to glyphosate in a number of crops.

Phenoxyacetic acid herbicides, such as 2,4-dichlorophenoxyacetic acid (2,4-D), show herbicidal activity towards dicotyledonous plants. Numerous 2,4-D-degrading bacterial strains have been isolated from soils exposed to 2,4-D (see, for example, Ka J. O., et al. (1994) Appl Environ Microbiol 60(4): 1106-15; Fulthorpe R. R., et al. (1995) Appl Environ Microbiol 61(9):3274-81). These bacteria produce a variety of enzymes involved in 2,4-D metabolism and detoxification. One such enzyme, 2,4-dichlorophenoxyacetate monooxygenase encoded by the tfdA gene from Alcaligenes eutrophus, metabolizes 2,4-D to non-phytotoxic 2,4-dichlorophenol. The tfda gene, or any other gene encoding a phenoxyacetic acid herbicide metabolizing activity, can be shuffled, either singly or with homologous sequences according to the methods described herein, to optimize nucleic acids encoding an improved phenoxyacetic acid herbicide metabolizing activity, and used to confer phenoxyacetic acid herbicide (e.g., 2,4-D) selectivity to dicotyledonous crops such as soybeans.

Fulthorpe et al. (supra) suggest that extensive interspecies transfer of a variety of homologous degradative genes has been involved in the evolution of 2,4-D-degrading bacteria. This natural diversity may be exploited by employing, for example, whole genome shuffling formats as described below to evolve herbicide tolerance nucleic acids which involve uncharacterized 24-D metabolic enzymes and/or multienzyme pathways.

Other examples of bacterial degradative genes which confer or have the potential to confer crop selectivity to herbicides may be found, for example, in Subramanian (1997) and in Quinn J. P. (1990; Biotech. Adv. 8:321-333).

DNA Shuffling to Modify Herbicide Target Proteins

A. Shuffling of EPSPS Genes

Glyphosate herbicidal activity is manifested by inhibiting 5-enolpyruvylshikimate-3-phosphate synthase (EPSP synthase, or EPSPS), an enzyme that catalyzes an essential step of the plant aromatic amino acid biosynthetic pathway. EPSPS is termed the “target site” of glyphosate in plants. Genes coding for EPSPS can be shuffled to produce a library of recombinant nucleic acids. The library can be screened for a recombinant herbicide tolerance nucleic acid that encodes a modified protein that is inhibited by glyphosate to a lesser extent than a native plant EPSPS, yet is comparable to a native plant EPSPS with respect to other natural properties, such as kinetic properties for substrates phosphoenolpyruvate (PEP) and shikimate 3-phosphate (S3P). The recombinant herbicide tolerance nucleic acid is used to confer glyphosate selectivity to crops.

Genes coding for EPSPS are isolated from various plants, bacteria, yeast, or other organisms directly from a cDNA library (if commercially available) or from mRNA isolated from plants (Padgette (1987) Arch. Biochem. Biophys. 258: 564-573; Gasser C S et al. (1988) J. Biol Chem. 263: 4280-4289), from bacterial DNA or RNA, from yeast DNA or RNA, or from any other desired organism (See, Ausubel, Sambrook or Berger, supra, for a description of standard methods of making libraries, e.g., from bacteria and yeast). Genes coding for EPSP synthases from various sources, or fragments of those genes, may also be chemically synthesized using sequences available from sources such as the GenBank database. For example, primers for gene isolation can be designed from EPSPS sequences available from various plants, e.g., petunia and tomato. EPSPS genes from various plant or non-plant sources can be shuffled individually or as a family, cloned, and transformed into cells, such as an E. coli AroA⁻ strain (Padgette, 1987).

Similarly, bacterial EPSPS genes, which are a preferred source for starting material (or to design starting material) for the various shuffling procedures herein can be used. A variety of bacterial EPSPS genes are known, many which are found in GenBank. These include accession number X00557 (the E. coli AroA gene for EPSPS), accession number U82268 (the AroA gene for EPSPS from Shigella dysenteriae), accession number M10947 (the AroA gene for EPSPS from Salmonella typhimurium), accession number X82415 (the AroA gene for EPSPS from Klebsiela pneumoniae), accession number L46372 (the AroA gene for EPSPS from Yersina pestis), and Z14100 (the AroA gene for EPSPS from Pseudomonas multocida). In addition, homologous sequences can be isolated (particularly from non-pathogenic strains) using standard techniques, such as hybridization to DNA libraries or by PCR amplification using degenerate (or conserved) primers.

Functional clones can be identified by, e.g., replica plating transformants onto minimal media plates containing increasing amounts of glyphosate which are inhibitory or lethal to wild type bacteria (or to AroA⁻ bacteria). This process can be automated using, e.g., a Q-bot apparatus, described below. Lack of, or decreased, inhibition of EPSPS by glyphosate, and kinetic properties for the natural substrates (PEP and S3P), are quantitated and compared to those of wild type enzyme (preferably, to wild type enzyme(s) of the crop plant(s) in which herbicide selectivity is desired) using published assay methods (Padgette, 1987). Iterative shuffling can be carried out with the genes isolated from selected clones, for optimization of the desired properties. Those genes coding for EPSP enzymes that are less sensitive or insensitive to glyphosate, but with little or no difference in the kinetic properties for natural substrates as compared to a preferred crop EPSP enzyme, are used to confer selectivity to the herbicide in the preferred crop, or to a number of crops.

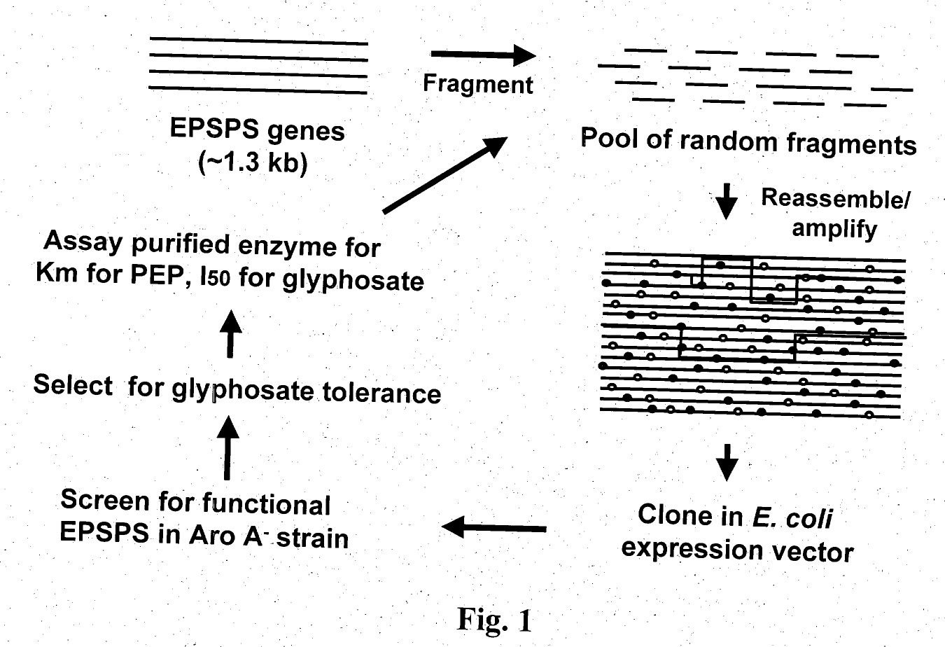

An exemplar family shuffling procedure for shuffling bacterial EPSPS genes for glyphosate tolerance is shown in FIG. 1. As depicted, EPSPS genes from bacteria (with an approximate average length of 1.3 kb) are fragmented, pooled, and reassembled/amplified. The resulting library of recombinant nucleic acids is cloned, transformed into an E. coli AroA⁻ strain, screened for EPSPS activity and selected for tolerance to increasing amounts of glyphosate. Enzyme can be purified from selected clones and analyzed for glyphosate-tolerant EPSPS activity with respect to kinetic parameters (e.g., Kᵢ for glyphosate and k_{cat}, Kₘ for substrates). Selected clones can be re-shuffled and the process iteratively repeated to further optimize kinetic parameters. Additional examples are provided in Examples 1 and 2 herein below.

B. Shuffling of Other Herbicide Target Genes

Acetolactate synthase (ALS; also known as acetohydroxyacid synthase or AHAS) is involved in the plant branched-chain amino acid biosynthetic pathway. ALS is inhibited by and is the target site for herbicides such as sulphonylureas, imidazolinones, and triazolopyrimidines. ALS sequences from Arabidopsis (GenBank accession T20822), cotton (GenBank accession Z46960), barley (GenBank accession AF059600) and other plant and non-plant sources are available and can be used to, e.g., synthesize nucleic acids for use as shuffling substrates, or as probes for isolation of ALS genes from other sources. DNA shuffling is employed, for example, in single gene or family shuffling formats as described herein to produce libraries which can be screened for ALS activities tolerant to one or more herbicides or classes of herbicides such as the sulphonylurea, imidazolinone, or triazolopyrimidine classes of herbicides, while retaining kinetic parameters comparable to those of a native plant ALS for natural substrates and cofactors.

Inhibition of the enzyme protoporphyrinogen oxidase (protox) in plant and green algal cells causes massive protoporphyrin IX accumulation, resulting in membrane deterioration and cell lethality in the light. Protox is the molecular target of herbicides including diphenyl ether-type herbicides. Protox sequences available in GenBank include those from Arabidopsis (GenBank accession D83139), the photosynthetic alga Chlamydomonas reinhardtii (GenBank accession AF068635), and tobacco (GenBank accession Y13465), which can be used as parental shuffling substrates and/or used find homologous protox sequences, e.g. by database searching or by probing cDNA libraries. DNA shuffling is employed to produce libraries which can be screened to recombinant herbicide tolerance nucleic acids encoding protox activities tolerant to diphenyl ether herbicides. For example, libraries of shuffled protox nucleic acids can be introduced into Chlamydomonas (Rochaix J D (1995) Ann. Rev. Genet. 29:209-230) and screened for tolerance activity to diphenyl ether herbicides (Randolph-Anderson B L et al. (1998) Plant Mol Biol 38:839-59).

DNA Shuffling to Evolve New Herbicide Tolerance Activities

In another general strategy, DNA shuffling is applied to genes or gene families to acquire new activities which mimic those of native plant herbicide target proteins. The candidate parent genes for shuffling encode proteins having functional and/or structural similarities to the native target protein, and lack, or have reduced, susceptibility to herbicide inhibition compared to the native target protein. Such genes are optimized by DNA shuffling, optionally together with nucleic acids derived from the target protein gene, to encode novel proteins which can functionally substitute for the native herbicide-sensitive target proteins in the plant.

The bacterial MurA gene encodes a UDP-N-acetylglucosamine enolpyruvyltransferase (EPT), which catalyzes the transfer of the enolpyruvyl moiety of phosphoenolpyruvate (PEP) to the 3-hydroxyl of UDP-N-acetylglucosamine. EPT is the only known enzyme other than EPSPS that catalyses the transfer of the enolpyruvate moiety of PEP to an acceptor substrate (Wanke C. et al. (1992) FEBS Lett. 310:271-276); however, unlike EPSPS, EPT is not inhibited by (i.e., is tolerant to) glyphosate. EPT has a very similar tertiary structure to that of EPSPS, despite an overall amino acid sequence identity of only 25% (Schonbrun E. et al. (1996) Structure 4(9):1065-1075).

DNA shuffling can be utilized to evolve MurA nucleic acids to encode a novel EPT derivative (denoted EPTD) which catalyses enolpyruvyl transfer to S3P and retains tolerance to glyphosate. The novel EPTD gene encodes an activity that can functionally substitute for EPSPS activity in the plant aromatic amino acid biosynthetic pathway, and thus confers glyphosate tolerance to plants containing the EPTD gene.

Sequences coding for EPT, or fragments thereof, are isolated from bacteria or other organisms directly from a commercially-available cDNA, or by making a cDNA library from bacterial DNA or RNA (or from any other desired organism) using standard methods, or can be chemically synthesized. A variety of bacterial EPT genes are known, including several found in GenBank. These include accession number M76452 (the E. coli MurA gene for EPT), accession number Z11835 (the gene from Enterobacter cloacae), accession number AF142781 (the MurA gene from Chlamydia trachomatis), and accession number X96711 (the MurA gene from Mycobacterium tuberculosis). Other homologous sequences can be identified from sequence repositories, or isolated using standard techniques such as hybridization to DNA libraries, PCR, or RT-PCR, using degenerate or conserved primers.

Libraries of shuffled EPT nucleic acids can be prepared following the techniques described herein. Inclusion of EPSPS-derived sequences in the shuffling reactions, particularly sequences derived from the S3P binding region, can facilitate evolution of EPT towards EPSPS-like specificity for the shikimate-3-phosphate acceptor. Shuffled libraries can be screened for glyphosate tolerance and the emergence of enolpyruvyl-shikimate phosphate synthesis activity as described in the previous section, from which candidate EPTD genes can be selected. Iterative shuffling can be carried out on the candidate EPTD genes, optionally with EPSPS sequences included, for optimization of substrate kinetic properties toward those of native plant EPSPS enzymes. Optimized herbicide tolerance nucleic acids encoding the novel EPTD enzymes can be introduced into a plant to confer glyphosate tolerance to the plant.

Automation of Screening

In screening it is advantageous to an assay that can be dependably used to identify a few mutants out of thousands that have potentially subtle increases in herbicide tolerance activity. The limiting factor in many assay formats is the uniformity of library cell (or viral) growth. This variation is the source of baseline variability in subsequent assays. Inoculum size and culture environment (temperaturelhumidity) are sources of cell growth variation. Automation of all aspects of establishing initial cultures and state-of-the-art temperature and humidity controlled incubators are useful in reducing variability.

In one aspect, library members in, e.g., cells, viral plaques, spores or the like, are separated on solid media to produce individual colonies (or plaques). Using an automated colony picker (e.g., the Q-bot, Genetix, U.K.), colonies are identified, picked, and 10,000 different mutants inoculated into 96 well microtiter dishes containing two 3 mm balls/well. The Q-bot does not pick an entire colony but rather inserts a pin through the center of the colony and exits with a small sampling of cells, (or mycelia) and spores (or viruses in plaque applications). The time the pin is in the colony, the number of dips to inoculate the culture medium, and the time the pin is in that medium each effect inoculum size, and each can be controlled and optimized. The uniform process of the Q-bot decreases human handling error and increases the rate of establishing cultures (roughly 10,000/4 hours). These cultures are then shaken in a temperature and humidity controlled incubator. The balls in the microtiter plates, which can be made of glass, steel, or other suitable inert substance, act to promote uniform aeration of cells and the dispersal of cellular materials similar to the blades of a fermentor. Steel balls are preferred as they can be manipulated using magnets.

The chance of finding the library component encoding an improved herbicide tolerance activity is increased by the number of individual mutants that can be screened by the assay. To increase the chances of identifying a pool of sufficient size, a prescreen that increases the number of mutants processed by about 10-fold can be used. Pools showing significant herbicide tolerance activity can be deconvoluted (e.g., cloned by limiting dilution) to identify single clones with the desired activity.

Formats for Sequence Recombination

The methods of the invention entail performing recombination (“shuffling”) and screening or selection to “evolve” individual genes, whole plasmids or viruses, multigene clusters, or even whole genomes (Stemmer (1995) Bio/Technology 13:549-553). Reiterative cycles of recombination and screening/selection can be performed to further evolve the nucleic acids of interest. Such techniques do not require the extensive analysis and computation required by conventional methods for polypeptide engineering. Shuffling allows the recombination of large numbers of mutations in a minimum number of selection cycles, in contrast to natural pairwise recombination events (e.g., as occur during sexual replication). Thus, the sequence recombination techniques described herein provide particular advantages in that they provide recombination between mutations in any or all of these, thereby providing a very fast way of exploring the manner in which different combinations of mutations can affect a desired result. In some instances, however, structural and/or functional information is available which, although not required for sequence recombination, provides opportunities for modification of the technique.

Exemplary formats and examples for sequence recombination, referred to, e.g., as “DNA shuffling,” “fast forced evolution,” or “molecular breeding,” have been described in the following patents and patent applications: U.S. Pat. No. 5,605,793; PCT Application WO 95/22625 (Serial No. PCT/US95/02126), filed Feb. 17, 1995; U.S. Ser. No. 08/425,684, filed Apr. 18, 1995; U.S. Ser. No. 08/621,430, filed Mar. 25, 1996; PCT Application WO 97/20078 (Serial No. PCT/US96/05480), filed Apr. 18, 1996; PCT Application WO 97/35966, filed Mar. 20, 1997; U.S. Ser. No. 08/675,502, filed Jul. 3, 1996; U.S. Ser. No. 08/721,824, filed Sep. 27, 1996; PCT Application WO 98/13487, filed Sep. 26, 1997; PCT Application WO 98/42832, filed Mar. 25, 1998; PCT Application WO 98/31837, filed Jan. 16, 1998; U.S. Ser. No. 09/166,188, filed Jul. 15, 1998; U.S. Ser. No. 09/354,922, filed Jul. 15, 1999; U.S. Ser. No. 60/118,813, filed Feb. 5, 1999; U.S. Ser. No. 60/141,049 filed Jun. 24, 1999; Stemmer, Science 270:1510 (1995); Stemmer et al., Gene 164:49-53 (1995); Stemmer, Bio/Technology 13:549-553 (1995); Stemmer, Proc. Natl. Acad. Sci. U.S.A. 91:10747-10751 (1994); Stemmer, Nature 370:389-391 (1994); Crameri et al., Nature Medicine 2(1):1-3 (1996); and Crameri et al., Nature Biotechnology 14:315-319 (1996), each of which is incorporated by reference in its entirety for all purposes.

The breeding procedure starts with at least two substrates that generally show substantial sequence identity to each other (ie., at least about 30%, 50%, 70%, 80% or 90% sequence identity), but differ from each other at certain positions. The difference can be any type of mutation, for example, substitutions, insertions and deletions. Often, different segments differ from each other in about 5-20 positions. For recombination to generate increased diversity relative to the starting materials, the starting materials must differ from each other in at least two nucleotide positions. That is, if there are only two substrates, there should be at least two divergent positions. If there are three substrates, for example, one substrate can differ from the second at a single position, and the second can differ from the third at a different single position. The starting DNA segments can be natural variants of each other, for example, allelic or species variants. The segments can also be from nonallelic genes showing some degree of structural and usually functional relatedness (e.g., different genes within a superfamily, such as the cytochrome P450 super family). The starting DNA segments can also be induced variants of each other. For example, one DNA segment can be produced by error-prone PCR replication of the other, or by substitution of a mutagenic cassette. Induced mutants can also be prepared by propagating one (or both) of the segments in a mutagenic strain. In these situations, strictly speaking, the second DNA segment is not a single segment but a large family of related segments. The different segments forming the starting materials are often the same length or substantially the same length. However, this need not be the case; for example; one segment can be a subsequence of another. The segments can be present as part of larger molecules, such as vectors, or can be in isolated form.

The starting DNA segments are recombined by any of the sequence recombination formats provided herein to generate a diverse library of recombinant DNA segments. Such a library can vary widely in size from having fewer than 10 to more than 10⁵, 10⁹, 10¹² or more members. In some embodiments, the starting segments and the recombinant libraries generated will include full-length coding sequences and any essential regulatory sequences, such as a promoter and polyadenylation sequence, required for expression. In other embodiments, the recombinant DNA segments in the library can be inserted into a common vector providing sequences necessary for expression before performing screening/selection.

Use of Restriction Enzyme Sites to Recombine Mutations

In some situations it is advantageous to use restriction enzyme sites in nucleic acids to direct the recombination of mutations in a nucleic acid sequence of interest. These techniques are particularly preferred in the evolution of fragments that cannot readily be shuffled by existing methods due to the presence of repeated DNA or other problematic primary sequence motifs. These situations also include recombination formats in which it is preferred to retain certain sequences unmutated. The use of restriction enzyme sites is also preferred for shuffling large fragments (typically greater than 10 kb), such as gene clusters that cannot be readily shuffled and “PCR-amplified” because of their size. Although fragments up to 50 kb have been reported to be amplified by PCR (Barnes, Proc. Natl. Acad. Sci. U.S.A. 91:2216-2220 (1994)), it can be problematic for fragments over 10 kb, and thus alternative methods for shuffling in the range of 10-50 kb and beyond are preferred. Preferably, the restriction endonucleases used are of the Class II type (Sambrook, Ausubel and Berger, supra) and of these, preferably those which generate nonpalindromic sticky end overhangs such as Alwn I, Sfi I or BstX1. These enzymes generate nonpalindromic ends that allow for efficient ordered reassembly with DNA ligase. Typically, restriction enzyme (or endonuclease) sites are identified by conventional restriction enzyme mapping techniques (Sambrook, Ausubel, and Berger, supra.), by analysis of sequence information for that gene, or by introduction of desired restriction sites into a nucleic acid sequence by synthesis (i.e. by incorporation of silent mutations).

The DNA substrate molecules to be digested can either be from in vivo replicated DNA, such as a plasmid preparation, or from PCR amplified nucleic acid fragments harboring the restriction enzyme recognition sites of interest, preferably near the ends of the fragment. Typically, at least two variants of a gene of interest, each having one or more mutations, are digested with at least one restriction enzyme determined to cut within the nucleic acid sequence of interest. The restriction fragments are then joined with DNA ligase to generate full length genes having shuffled regions. The number of regions shuffled will depend on the number of cuts within the nucleic acid sequence of interest. The shuffled molecules can be introduced into cells as described above and screened or selected for a desired property as described herein. Nucleic acid can then be isolated from pools (libraries), or clones having desired properties and subjected to the same procedure until a desired degree of improvement is obtained.

In some embodiments, at least one DNA substrate molecule or fragment thereof is isolated and subjected to mutagenesis. In some embodiments, the pool or library of religated restriction fragments are subjected to mutagenesis before the digestion-ligation process is repeated. “Mutagenesis” as used herein comprises such techniques known in the art as PCR mutagenesis, oligonucleotide-directed mutagenesis, site-directed mutagenesis, etc., and recursive sequence recombination by any of the techniques described herein.

Reassembly PCR

A further technique for recombining mutations in a nucleic acid sequence utilizes “reassembly PCR.” This method can be used to assemble multiple segments that have been separately evolved into a full length nucleic acid template such as a gene. This technique is performed when a pool of advantageous mutants is known from previous work or has been identified by screening mutants that may have been created by any mutagenesis technique known in the art, such as PCR mutagenesis, cassette mutagenesis, doped oligo mutagenesis, chemical mutagenesis, or propagation of the DNA template in vivo in mutator strains. Boundaries defining segments of a nucleic acid sequence of interest preferably lie in intergenic regions, introns, or areas of a gene not likely to have mutations of interest. Preferably, oligonucleotide primers (oligos) are synthesized for PCR amplification of segments of the nucleic acid sequence of interest, such that the sequences of the oligonucleotides overlap the junctions of two segments. The overlap region is typically about 10 to 100 nucleotides in length. Each of the segments is amplified with a set of such primers. The PCR products are then “reassembled” according to assembly protocols such as those discussed herein to assemble randomly fragmented genes. In brief, in an assembly protocol the PCR products are first purified away from the primers, by, for example, gel electrophoresis or size exclusion chromatography. Purified products are mixed together and subjected to about 1-10 cycles of denaturing, reannealing, and extension in the presence of polymerase and deoxynucleoside triphosphates (dNTP's) and appropriate buffer salts in the absence of additional primers (“self-priming”). Subsequent PCR with primers flanking the gene are used to amplify the yield of the fully reassembled and shuffled genes.

In some embodiments, the resulting reassembled genes are subjected to mutagenesis before the process is repeated.

In a further embodiment, the PCR primers for amplification of segments of the nucleic acid sequence of interest are used to introduce variation into the gene of interest as follows. Mutations at sites of interest in a nucleic acid sequence are identified by screening or selection, by sequencing homologues of the nucleic acid sequence, and so on. Oligonucleotide PCR primers are then synthesized which encode wild type or mutant information at sites of interest. These primers are then used in PCR mutagenesis to generate libraries of full length genes encoding permutations of wild type and mutant information at the designated positions. This technique is typically advantageous in cases where the screening or selection process is expensive, cumbersome, or impractical relative to the cost of sequencing the genes of mutants of interest and synthesizing mutagenic oligonucleotides.

Site Directed Mutagenesis (SDM) with Oligonucleotides Encoding Homologue Mutations Followed by Shuffling

In some embodiments of the invention, sequence information from one or more substrate sequences is added to a given “parental” sequence of interest, with subsequent recombination between rounds of screening or selection. Typically, this is done with site-directed mutagenesis performed by techniques well known in the art (e.g., Berger, Ausubel and Sambrook, supra.) with one substrate as template and oligonucleotides encoding single or multiple mutations from other substrate sequences, e.g. homologous genes. After screening or selection for an improved phenotype of interest, the selected recombinant(s) can be further evolved using RSR techniques described herein. After screening or selection, site-directed mutagenesis can be done again with another collection of oligonucleotides encoding homologue mutations, and the above process repeated until the desired properties are obtained.

When the difference between two homologues is one or more single point mutations in a codon, degenerate oligonucleotides can be used that encode the sequences in both homologues. One oligonucleotide can include many such degenerate codons and still allow one to exhaustively search all permutations over that block of sequence.

When the homologue sequence space is very large, it can be advantageous to restrict the search to certain variants. Thus, for example, computer modeling tools (Lathrop et al. (1996) J. Mol. Biol., 255: 641-665) can be used to model each homologue mutation onto the target protein and discard any mutations that are predicted to grossly disrupt structure and function.

In Vitro DNA Shuffling Formats

In one embodiment for shuffling DNA sequences in vitro, the initial substrates for recombination are a pool of related sequences, e.g., different, variant forms, as homologs from different individuals, strains, or species of an organism, or related sequences from the same organism, as allelic variations. The sequences can be DNA or RNA and can be of various lengths depending on the size of the gene or DNA fragment to be recombined or reassembled. Preferably the sequences are from 50 base pairs (bp) to 50 kilobases (kb).

The pool of related substrates are converted into overlapping fragments, e.g., from about 5 bp to 5 kb or more. Often, for example, the size of the fragments is from about 10 bp to 1000 bp, and sometimes the size of the DNA fragments is from about 100 bp to 500 bp. The conversion can be effected by a number of different methods, such as DNase I or RNase digestion, random shearing or partial restriction enzyme digestion. For discussions of protocols for the isolation, manipulation, enzymatic digestion, and the like of nucleic acids, see, for example, Sambrook et al. and Ausubel, both supra. The concentration of nucleic acid fragments of a particular length and sequence is often less than 0.1% or 1% by weight of the total nucleic acid. The number of different specific nucleic acid fragments in the mixture is usually at least about 100, 500 or 1000.

The mixed population of nucleic acid fragments are converted to at least partially single-stranded form using a variety of techniques, including, for example, heating, chemical denaturation, use of DNA binding proteins, and the like. Conversion can be effected by heating to about 80° C. to 100° C., more preferably from 90° C. to 96° C., to form single-stranded nucleic acid fragments and then reannealing. Conversion can also be effected by treatment with single-stranded DNA binding protein (see Wold (1997) Annu. Rev. Biochem. 66:61-92) or recA protein (see, e.g., Kiianitsa (1997) Proc. Natl. Acad. Sci. USA 94:7837-7840). Single-stranded nucleic acid fragments having regions of sequence identity with other single-stranded nucleic acid fragments can then be reannealed by cooling to 20° C. to 75° C., and preferably from 40° C. to 65° C. Renaturation can be accelerated by the addition of polyethylene glycol (PEG), other volume-excluding reagents or salt. The salt concentration is preferably from 0 mM to 200 mM, more preferably the salt concentration is from 10 mM to 100 mM. The salt may be KCl or NaCl. The concentration of PEG is preferably from 0% to 20%, more preferably from 5% to 10%. The fragments that reanneal can be from different substrates. The annealed nucleic acid fragments are incubated in the presence of a nucleic acid polymerase, such as Taq or Klenow, and dNTP's (i.e. DATP, dCTP, dGTP and dTTP). If regions of sequence identity are large, Taq polymerase can be used with an annealing temperature of between 45-65° C. If the areas of identity are small, Klenow polymerase can be used with an annealing temperature of between 20-30° C. The polymerase can be added to the random nucleic acid fragments prior to annealing, simultaneously with annealing or after annealing.

The process of denaturation, renaturation and incubation in the presence of polymerase of overlapping fragments to generate a collection of polynucleotides containing different permutations of fragments is sometimes referred to as shuffling of the nucleic acid in vitro. This cycle is repeated for a desired number of times. Preferably the cycle is repeated from 2 to 100 times, more preferably the sequence is repeated from 10 to 40 times. The resulting nucleic acids are a family of double-stranded polynucleotides of from about 50 bp to about 100 kb, preferably from 500 bp to 50 kb. The population represents variants of the starting substrates showing substantial sequence identity thereto but also diverging at several positions. The population has many more members than the starting substrates. The population of fragments resulting from shuffling is used to transform host cells, optionally after cloning into a vector.

In one embodiment utilizing in vitro shuffling, subsequences of recombination substrates can be generated by amplifying the full-length sequences under conditions which produce a substantial fraction, typically at least 20 percent or more, of incompletely extended amplification products. Another embodiment uses random primers to prime the entire template DNA to generate less than full length amplification products. The amplification products, including the incompletely extended amplification products are denatured and subjected to at least one additional cycle of reannealing and amplification. This variation, in which at least one cycle of reannealing and amplification provides a substantial fraction of incompletely extended products, is termed “stuttering.”In the subsequent amplification round, the partially extended. (less than full length) products reanneal to and prime extension on different sequence-related template species. In another embodiment, the conversion of substrates to fragments can be effected by partial PCR amplification of substrates.

In another embodiment, a mixture of fragments is spiked with one or more oligonucleotides. The oligonucleotides can be designed to include precharacterized mutations of a wildtype sequence, or sites of natural variations between individuals or species. The oligonucleotides also include sufficient sequence or structural homology flanking such mutations or variations to allow annealing with the wildtype fragments. Annealing temperatures can be adjusted depending on the length of homology.

In a further embodiment, recombination occurs in at least one cycle by template switching, such as when a DNA fragment derived from one template primes on the homologous position of a related but different template. Template switching can be induced by addition of recA (see, Kiianitsa (1997) supra), rad51 (see, Namsaraev (1997) Mol. Cell. Biol. 17:5359-5368), rad55 (see, Clever (1997) EMBO J. 16:2535-2544), rad57 (see, Sung (1997) Genes Dev. 11:1111-1121) or, other polymerases (e.g., viral polymerases, reverse transcriptase) to the amplification mixture. Template switching can also be increased by increasing the DNA template concentration.

Another embodiment utilizes at least one cycle of amplification, which can be conducted using a collection of overlapping single-stranded DNA fragments of related sequence, and different lengths. Fragments can be prepared using a single stranded DNA phage, such as M13 (see, Wang (1997) Biochemistry 36:9486-9492). Each fragment can hybridize to and prime polynucleotide chain extension of a second fragment from the collection, thus forming sequence-recombined polynucleotides. In a further variation, ssDNA fragments of variable length can be generated from a single primer by Pfu, Taq, Vent, Deep Vent, UlTma DNA polymerase or other DNA polymerases on a first DNA template (see, Cline (1996) Nucleic Acids Res. 24:3546-3551). The single stranded DNA fragments are used as primers for a second, Kunkel-type template, consisting of a uracil-containing circular ssDNA. This results in multiple substitutions of the first template into the second. See, Levichkin (1995) Mol. Biology 29:572-577; Jung (1992) Gene 121:17-24.

In some embodiments of the invention, shuffled nucleic acids obtained by use of the recursive recombination methods of the invention, are put into a cell and/or organism for screening. Shuffled herbicide tolerance genes can be introduced into, for example, bacterial cells, yeast cells, or plant cells for initial screening. Bacillus species (such as B. subtilis) and E. coli are two examples of suitable bacterial cells into which one can insert and express shuffled herbicide tolerance genes. The shuffled genes can be introduced into bacterial or yeast cells either by integration into the chromosomal DNA or as plasmids. Shuffled genes can also be introduced into plant cells for screening purposes. Thus, a transgene of interest can be modified using the recursive sequence recombination methods of the invention in vitro and reinserted into the cell for in vivo/in situ selection for the new or improved property.

Oligonucleotide and in Silico Shuffling Formats

In addition to the formats for shuffling noted above, at least two additional related formats are useful in the practice of the present invention. The first, referred to as “in silico” shuffling utilizes computer algorithms to perform “virtual” shuffling using genetic operators in a computer. As applied to the present invention, herbicide tolerance nucleic acid sequence strings are recombined in a computer system and desirable products are made, e.g., by reassembly PCR of synthetic oligonucleotides. In silico shuffling is described in detail in a patent application entitled “METHODS FOR MAIG CHARACTER STRINGS, POLYNUCLEOTIDES & POLYPEPTIDES HAVING DESIRED CHARACTERISTICS” filed Feb. 5, 1999, U.S. Ser. No. 60/118,854. In brief, genetic operators (algorithms which represent given genetic events such as point mutations, recombination of two strands of homologous nucleic acids, etc.) are used to model recombinational or mutational events which can occur in one or more nucleic acid, e.g., by aligning nucleic acid sequence strings (using standard alignment software, or by manual inspection and alignment) and predicting recombinational outcomes. The predicted recombinational outcomes are used to produce corresponding molecules, e.g., by. oligonucleotide synthesis and reassembly PCR.

The second useful for-mat is referred to as “oligonucleotide mediated shuffling” in which oligonucleotides corresponding to a family of related homologous nucleic acids (e.g., as applied to the present invention, interspecific or allelic variants of a herbicide tolerance nucleic acid or a-potential herbicide tolerance nucleic acid) which are recombined to produce selectable nucleic acids. This format is described in detail in patent applications entitled “OLIGONULCLEOTIDE MEDIATED NUCLEIC ACID RECOMBINATION” filed Feb. 5, 1999 having U.S. Ser. No. 60/118,813, and filed Jun. 24, 1999 having U.S. Ser. No. 60/141,049. The technique can be used to recombine homologous or even non-homologous nucleic acid sequences.

One advantage of the oligonucleotide-mediated shuffling format is the ability to recombine homologous nucleic acids with low sequence similarity, or even non-homologous nucleic acids. In these low-homology oligonucleotide shuffling methods, one or more set of fragmented nucleic acids are recombined, e.g., with a with a set of crossover family diversity oligonucleotides. Each of these crossover oligonucleotides have a plurality of sequence diversity domains corresponding to a plurality of sequence diversity domains from homologous or non-homologous nucleic acids with low sequence similarity. The fragmented oligonucleotides, which are derived by comparison to one or more homologous or non-homologous nucleic acids, can hybridize to one or more region of the crossover oligos, facilitating recombination.

When recombining homologous nucleic acids, sets of overlapping family gene shuffling oligonucleotides (which are derived by comparison of homologous nucleic acids and synthesis of oligonucleotide fragments) are hybridized and elongated (e.g., by reassembly PCR), providing a population of recombined nucleic acids, which can be selected for a desired trait or property. Typically, the set of overlapping family shuffling gene oligonucleotides include a plurality of oligonucleotide member types which have consensus region subsequences derived from a plurality of homologous target nucleic acids.

Typically, family gene shuffling oligonucleotide are provided by aligning homologous nucleic acid sequences to select conserved regions of sequence identity and regions of sequence diversity. A plurality of family gene shuffling oligonucleotides are synthesized (serially or in parallel) which correspond to at least one region of sequence diversity.

Sets of fragments, or subsets of fragments used in oligonucleotide shuffling approaches can be provided by cleaving one or more homologous nucleic acids (e.g., with a DNase), or, more commonly, by synthesizing a set of oligonucleotides corresponding to a plurality of regions of at least one nucleic acid (typically oligonucleotides corresponding to a fill-length nucleic acid are provided as members of a set of nucleic acid fragments). In the shuffling procedures herein, these cleavage fragments (e.g., fragments of a potential herbicide tolerance gene) can be used in conjunction with family gene shuffling oligonucleotides, e.g., in one or more recombination reaction to produce recombinant herbicide tolerance nucleic acids.

Codon Modification Shuffling

Procedures for codon modification shuffling are described in detail in patent applications entitled “SHUFFLING OF CODON ALTERED GENES” filed Sep. 29, 1998 having U.S. Ser. No. 60/102,362, and filed Jan. 29, 1999 having U.S. Ser. No. 60/117,729. In brief, by synthesizing nucleic acids in which the codons which encode polypeptides are altered, it is possible to access a completely different mutational cloud upon subsequent mutation of the nucleic acid. This increases the sequence diversity of the starting nucleic acids for shuffling protocols, which alters the rate and results of forced evolution procedures. Codon modification procedures can be used to modify any herbicide tolerance (or potential herbicide tolerance) nucleic acid herein, e.g., prior to performing DNA shuffling, or codon modification approaches can be used in conjunction with Oligonucleotide Shuffling procedures as described supra.

In these methods, a first nucleic acid sequence encoding a first polypeptide sequence is selected. A plurality of codon altered nucleic acid sequences, each of which encode the first polypeptide, or a modified or related polypeptide, is then selected (e.g., a library of codon altered nucleic acids can be selected in a biological assay which recognizes library components or activities), and the plurality of codon-altered nucleic acid sequences is recombined to produce a target codon altered nucleic acid encoding a second protein. The target codon altered nucleic acid is then screened for a detectable functional or structural property, optionally including comparison to the properties of the first polypeptide and/or related polypeptides. The goal of such screening is to identify a polypeptide that has a structural or functional property equivalent or superior to the first polypeptide or related polypeptide. A nucleic acid encoding such a polypeptide can be used in essentially any procedure desired, including introducing the target codon altered nucleic acid into a cell, vector, virus, attenuated virus (e.g., as a component of a vaccine or immunogenic composition), transgenic organism, or the like.

In Vivo DNA Shuffling Formats

In some embodiments of the invention, DNA substrate molecules are introduced into cells, wherein the cellular machinery directs their recombination. For example, a library of mutants is constructed and screened or selected for mutants with improved phenotypes by any of the techniques described herein. The DNA substrate molecules encoding the best candidates are recovered by any of the techniques described herein, then fragmented and used to transfect a plant host and screened or selected for improved function. If further improvement is desired, the DNA substrate molecules are recovered from the plant host cell, such as by PCR, and the process is repeated until a desired level of improvement is obtained. In some embodiments, the fragments are denatured and reannealed prior to transfection, coated with recombination stimulating proteins such as recA, or co-transfected with a selectable marker such as NeoR to allow the positive selection for cells receiving recombined versions of the gene of interest. Methods for in vivo shuffling are described in, for example, PCT applications WO 98/13487 and WO 97/07205.

The efficiency of in vivo shuffling can be enhanced by increasing the copy number of a gene of interest in the host cells. For example, the majority of bacterial cells in stationary phase cultures grown in rich media contain two, four or eight genomes. In minimal medium the cells contain one or two genomes. The number of genomes per bacterial cell thus depends on the growth rate of the cell as it enters stationary phase. This is because rapidly growing cells contain multiple replication forks, resulting in several genomes in the cells after termination. The number of genomes is strain dependent, although all strains tested have more than one chromosome in stationary phase. The number of genomes in stationary phase cells decreases with time. This appears to be due to fragmentation and degradation of entire chromosomes, similar to apoptosis in mammalian cells. This fragmentation of genomes in cells containing multiple genome copies results in massive recombination and mutagenesis. The presence of multiple genome copies in such cells results in a higher frequency of homologous recombination in these cells, both between copies of a gene in different genomes within the cell, and between a genome within the cell and a transfected fragment. The increased frequency of recombination allows one to evolve a gene evolved more quickly to acquire optimized characteristics.

In nature, the existence of multiple genomic copies in a cell type would usually not be advantageous due to the greater nutritional requirements needed to maintain this copy number. However, artificial conditions can be devised to select for high copy number. Modified cells having recombinant genomes are grown in rich media (in which conditions, multicopy number should not be a disadvantage) and exposed to a mutagen, such as ultraviolet or gamma irradiation or a chemical mutagen, e.g., mitomycin, nitrous acid, photoactivated psoralens, alone or in combination, which induces DNA breaks amenable to repair by recombination. These conditions select for cells having multicopy number due to the greater efficiency with which mutations can be excised. Modified cells surviving exposure to mutagen are enriched for cells with multiple genome copies. If desired, selected cells can be individually analyzed for genome copy number (e.g., by quantitative hybridization with appropriate controls). For example, individual cells can be sorted using a cell sorter for those cells containing more DNA, e.g., using DNA specific fluorescent compounds or sorting for increased size using light dispersion. Some or all of the collection of cells surviving selection are tested for the presence of a gene that is optimized for the desired property.

In one embodiment, phage libraries are made and recombined in mutator strains such as cells with mutant or impaired gene products of mutS, mutT, muth, mutL, ovrD, dcm, vsr, umuC, umuD, sbcB, recJ, etc. The impairment is achieved by genetic mutation, allelic replacement, selective inhibition by an added reagent such as a small compound or an expressed antisense RNA, or other techniques. High multiplicity of infection (MOI) libraries are used to infect the cells to increase recombination frequency.

Additional strategies for making phage libraries and or for recombining DNA from donor and recipient cells are set forth in U.S. Pat. No. 5,521,077. Additional recombination strategies for recombining plasmids in yeast are set forth in PCT application WO 97/07205.

Whole Genome Shuffling

In one embodiment, the selection methods herein are utilized in a “whole genome shuffling” format. An extensive guide to the many forms of whole genome shuffling is found in applications entitled “EVOLUTION OF WHOLE CELLS AND ORGANISMS BY RECURSIVE SEQUENCE RECOMBINATION”, filed Jul. 15, 1998 having U.S. Ser. No. 09/166,188, and filed Jul. 15, 1999 having U.S. Ser. No. 09/354,922.

In brief, whole genome shuffling makes no presuppositions at all regarding what nucleic acids may confer a desired property. Instead, entire genomes (e.g., from a genomic library, or isolated from an organism) are shuffled in cells and selection protocols applied to the cells.

Methods of evolving a cell to acquire a desired function by whole genome shuffling entail, e.g., introducing a library of DNA fragments into a plurality of cells, whereby at least one of the fragments undergoes recombination with a segment in the genome or an episome of the cells to produce modified cells. Optionally, these modified cells are bred to increase the diversity of the resulting recombined cellular population. The modified cells, or the recombined cellular population, are then screened for modified or recombined cells that have evolved toward acquisition of the desired function. DNA from the modified cells that have evolved toward the desired function is then optionally recombined with a further library of DNA fragments, at least one of which undergoes recombination with a segment in the genome or the episome of the modified cells to produce further modified cells. The further modified cells are then screened for further modified cells that have further evolved toward acquisition of the desired function. Steps of recombination and screening/selection are repeated as required until the further modified cells have acquired the desired function. In one variation of the method, modified cells are recursively recombined to increase diversity of the cells prior to performing any selection steps on any resulting cells.

An application of recursive whole genome shuffling is the evolution of plant cells, and transgenic plants derived from the same, to acquire tolerance to herbicides. The substrates for recombination can be, e.g., whole genomic libraries, fractions thereof or focused libraries containing variants of gene(s) known or suspected to confer tolerance to one of the above agents. Frequently, library fragments are obtained from a different species to the plant being evolved. Regardless of the precise shuffling methodology used, the screening and selection methods described above, including selection for tolerance activity to dicamba, bisphosphonate, sulfentrazone, an imidazolinone, a sulfonylurea, a triazolopyrimidine or the like, can be performed as discussed herein.

The DNA fragments are introduced into plant tissues, cultured plant cells or plant protoplasts by standard methods including electroporation (From et al. (1985) Proc. Natl. Acad. Sci. USA 82:5824), infection by viral vectors such as cauliflower mosaic virus (CaMV; Hohn et al., Molecular Biology of Plant Tumors (Academic Press, New York, 1982) pp. 549-560; Howell, U.S. Pat. No. 4,407,956), high velocity ballistic penetration by small particles with the nucleic acid either within the matrix of small beads or particles, or on the surface (Klein et al. (1987) Nature 327:70-73), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233:496-498; Fraley et al. (1983) Proc. Natl. Acad. Sci. USA 80:4803).

Diversity can also be generated by genetic exchange between plant protoplasts. Procedures for formation and fusion of plant protoplasts are described by Takahashi et al., U.S. Pat. No. 4,677,066; Akagi et al., U.S. Pat. No. 5,360,725; Shimamoto et al., U.S. Pat. No.5,250,433; Cheney et al., U.S. Pat. No.5,426,040.

After a suitable period of incubation to allow recombination to occur and for expression of recombinant genes, the plant cells are contacted with the herbicide to which tolerance is to be acquired, and surviving plant cells are collected. Some or all of these plant cells can be subject to a further round of recombination and screening. Eventually, plant cells having the required degree of tolerance are obtained.

These cells can then be cultured into transgenic plants. Plant regeneration from cultured protoplasts is described in Evans et al., “Protoplast Isolation and Culture,” Handbook of Plant Cell Cultures 1, 124-176 (MacMillan Publishing Co., New York, 1983); Davey, “Recent Developments in the Culture and Regeneration of Plant Protoplasts,” Protoplasts, (1983) pp. 12-29, (Birkhauser, Basal 1983); Dale, “Protoplast Culture and Plant Regeneration of Cereals and Other Recalcitrant Crops,” Protoplasts (1983) pp. 31-41, (Birkhauser, Basel 1983); Binding, “Regeneration of Plants,” Plant Protoplasts, pp. 21-73, (CRC Press, Boca Raton, 1985) and other references available to persons of skill. Additional details regarding plant regeneration from cells are also found below.

In a variation of the above method, one or more preliminary rounds of recombination and screening can be performed in bacterial cells according to the same general strategy as described for plant cells. More rapid evolution can be achieved in bacterial cells due to their greater growth rate and the greater efficiency with which DNA can be introduced into such cells. After one or more rounds of recombination/screening, a DNA fragment library is recovered from bacteria and transformed into the plants. The library can either be a complete library or a focused library. A focused library can be produced by amplification from primers specific for plant sequences, particularly plant sequences known or suspected to have a role in conferring tolerance.

Plant genome shuffling allows recursive cycles to be used for the introduction and recombination of genes or pathways that confer improved properties to desired plant species. Any plant species, including weeds and wild cultivars, showing a desired trait, such as herbicide tolerance, can be used as the source of DNA that is introduced into the crop or horticultural host plant species.

Genomic DNA prepared from the source plant is fragmented (e.g. by DNaseI, restriction enzymes, or mechanically) and cloned into a vector suitable for making plant genomic libraries, such as pGA482 (An. G. (1995) Methods Mol. Biol. 44:47-58). This vector contains the A. tumefaciens left and right borders needed for gene transfer to plant cells and antibiotic markers for selection in E. coli, Agrobacterium, and plant cells. A multicloning site is provided for insertion of the genomic fragments. A cos sequence is present for the efficient packaging of DNA into bacteriophage lambda heads for transfection of the primary library into E. coli. The vector accepts DNA fragments of 25-40 kb.

The primary library can also be directly electroporated into an A. tumefaciens or A. rhizogenes strain that is used to infect and transform host plant cells (Main, G D et al. (1995) Methods Mol. Biol. 44:405-412). Alternatively, DNA can be introduced by electroporation or PEG-mediated uptake into protoplasts of the recipient plant species (Bilang et al. (1994) Plant Mol. Biol Manual, Kluwer Academic Publishers, A1:1-16) or by particle bombardment of cells or tissues (Christou, ibid., A2:1-15). If necessary, antibiotic markers in the T-DNA region can be eliminated, as long as selection for the trait is possible, so that the final plant products contain no antibiotic genes.

Stably transformed whole cells acquiring the trait are selected on solid or liquid media containing the herbicide to which the introduced DNA confers tolerance. If the trait in question cannot be selected for directly, transformed cells can be selected with antibiotics and allowed to form callus or regenerated to whole plants and then screened for the desired property.

The second and further cycles consist of isolating genomic DNA from each transgenic line and introducing it into one or more of the other transgenic lines. In each round, transformed cells are selected or screened, typically in an incremental fashion (increasing dosages, etc.). To speed the process of using multiple cycles of transformation, plant regeneration can be eliminated until the last round. Callus tissue generated from the protoplasts or transformed tissues can serve as a source of genomic DNA and new host cells. After the final round, fertile plants are regenerated and the progeny are selected for homozygosity of the inserted DNAs. Alternatively, microspores can be isolated as homozygotes generated from spontaneous diploids. Ultimately, a new plant is created that carries multiple inserts which additively or synergistically combine to confer high levels of the desired trait.

In addition, the introduced DNA that confers the desired trait can be traced because it is flanked by known sequences in the vector. Either PCR or plasmid rescue is used to isolate the sequences and characterize them in more detail. Long PCR (Foord, O S and Rose, E A, 1995, PCR Primer: A Laboratory Manual, CSHL Press, pp 63-77) of the full 25-40 kb insert is achieved with the proper reagents and techniques using as primers the T-DNA border sequences. If the vector is modified to contain the E. coli origin of replication and an antibiotic marker between the T-DNA borders, a rare cutting restriction enzyme, such as NotI or SfiI, that cuts only at the ends of the inserted DNA is used to create fragments containing the source plant DNA that are then self-ligated and transformed into E. coli where they replicate as plasmids. The total DNA or subfragment of it that is responsible for the transferred trait can be subjected to in vitro evolution by DNA shuffling. The shuffled library is then introduced into host plant cells and screened for improvement of the trait. In this way, single and multigene traits can be transferred from one species to another and optimized for higher expression or activity leading to whole organism improvement.

Alternatively, the cells can be transformed microspores with the regenerated haploid plants being screened directly for improved traits. Microspores are haploid (In) male spores that develop into pollen grains. Anthers contain a large numbers of microspores in early-uninucleate to first-mitosis stages. Microspores have been successfully induced to develop into plants for most species, such as, e.g., rice (Chen, C C (1977) In Vitro. 13: 484-489), tobacco (Atanassov, I. et al. (1998) Plant Mol Biol. 38:1169-1178), Tradescantia (Savage J R K and Papworth D G. (1998) Mutat Res. 422:313-322), Arabidopsis (Park S K et al. (1998) Development. 125:3789-3799), sugar beet (Majewska-Sawka A and Rodrigues-Garcia M I (1996) J. Cell Sci. 109:859-866), barley (Olsen F L (1991) Hereditas 115:255-266), and oilseed rape (Boutillier K A et al. (1994) Plant Mol Biol. 26:1711-1723).

The plants derived from microspores are predominantly haploid or diploid (infrequently polyploid and aneuploid). The diploid plants are homozygous and fertile and can be generated in a relatively short time. Microspores obtained from F1 hybrid plants represent great diversity, thus being an excellent model for studying recombination. In addition, microspores can be transformed with T-DNA introduced by Agrobacterium or other available means and then regenerated into individual plants. Protoplasts can be made from microspores and can be fused by methods known in the art.

Protoplasts generated from microspores (especially the haploid ones) are pooled and fused. Microspores obtained from plants generated by protoplast fusion are pooled and fused again, increasing the genetic diversity of the resulting microspores. Microspores can be subjected to mutagenesis in various ways, such as by chemical mutagenesis, radiation-induced mutagenesis and, e.g., t-DNA transformation, prior to fusion or regeneration. New mutations which are generated can be recombined through the recursive processes described above and herein.

Rapid Evolution of Herbicide Tolerance Activity in Whole Cells

Whole genome shuffling methods such as those discussed above can be used to evolve plant cells having distinct or improved herbicide tolerance activities compared to the parental plant cell(s). This method is particularly useful in cases where a gene which confers tolerance to a particular herbicide or a mechanism by which tolerance to a particular herbicide is conferred is not known, or where several alternative tolerance mechanisms are known and/or can be envisaged. The plant cells chosen to receive foreign DNA fragments are preferably from crop species. Foreign DNA for transformation can be isolated from a different plant species, preferably one that is tolerant to the herbicide, or from other organisms, particularly organisms which posses known or suspected herbicide tolerance activities. DNA is isolated by standard methods (Sambrook, 1989) and fragmented, e.g. by shearing. The DNA is introduced into a population of protoplasts or cells in suspension culture. The population is then subjected to a dose of the herbicide that kills a large portion, for example 95%, of the cells. Survivors are subjected to further rounds of transformation, either with donor DNA or DNA from the surviving pool. The process continues recursively until the desired level of tolerance is attained. Plants are then regenerated from the evolved cells or protoplasts, and the tolerance trait(s) bred into elite lines. A further refinement of this method is attained if the DNA fragments used in the transformation contain specific sequences that enable the incorporated DNA to be recovered from the transformed plant by PCR. In this manner, recombinant nucleic acids encoding herbicide tolerance activities can be transferred into any species, not just the one in which the transformation and selection were carried out.

The use of certain existing commercially important herbicides could be extended into new applications if appropriate crop selectivity could be obtained. Among such herbicides, for example, are those of the chloroacetamide class, such as metolachlor, acetochlor and dimethenamid. The mode of action of the chloroacetamides is unknown and tolerance to herbicides of this class has not been observed. The method described above could be used to evolve cereal crop plant cells to acquire tolerance to chloroacetamide herbicides. The cells could then be regenerated into chloroacetamide-selective crops, upon which chloroacetamide herbicides could be used, for example, as a pre-emergence treatment for grass weeds.

As an example, plant cells can be evolved to acquire tolerance to an herbicide that blocks photosynthesis, such as one that inhibits photosystem II (including phenylcarbamates, pyridazinones, triazines, triazinones, uracils, and the like) by introducing DNA fragments from isolates of the green photosynthetic alga Chlamydomonas reinhardtii that are tolerant to the herbicide (see, e.g., Erickson J M et al. (1989) Plant Cell 1(3):361-71.

In another example, plant cells can be evolved to acquire tolerance to the herbicide hydantocidin, which kills all species of plants. Hydantocidin is phosphorylated in plants by an unknown mechanism. The phosphorylated product inhibits adenylosuccinate synthetase, an enzyme in the purine biosynthesis pathway. Hydantocidin lacking the phosphate group does not inhibit the enzyme. Although adenylosuccinate synthetase from E. coli and rat liver is inhibited by phosphorylated hydantocidin equally as well as the plant enzyme, hydantocidin itself is minimally toxic to these organisms. Possible mechanisms which reduce the toxicity of hydantocidin in these organisms as compared to plant cells include reduced uptake of hydantocidin, reduced phosphorylation of hydantocidin, or increased de-phosphorylation of the toxic phospho-hydantocidin, among others. By whole genome shuffling methods described above, using DNA fragments isolated from genomes of organisms (such as bacteria) in which hydantocidin is minimally toxic or non-toxic, evolution of plant cells for tolerance to hydantocidin can be accomplished.

Making Transgenic Plants

In one aspect, nucleic acids shuffled for herbicide tolerance by any of the techniques noted above are used to make transgenic plant cells. In another aspect, the nucleic acids are used to make transgenic plants, thereby providing transgenic plants.

The transformation of plant cells and protoplasts in accordance with the invention may be carried out in essentially any of the various ways known to those skilled in the art of plant molecular biology, including, but not limited to, the methods described herein. See, in general, Methods in Enzymology Vol. 153 (“Recombinant DNA Part D”) 1987, Wu and Grossman Eds., Academic Press, incorporated herein by reference. As used herein, the term “transformation” means alteration of the genotype of a host plant by the introduction of a nucleic acid sequence, i e., a “foreign” nucleic acid sequence. The foreign nucleic acid sequence need not necessarily originate from a different source, but it will, at some point, have been external to the cell into which it is to be introduced.

In addition to Berger, Ausubel and Sambrook, useful general references for plant cell cloning, culture and regeneration include Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods, Springer Lab Manual, Springer-Verlag (Berlin Heidelberg New York) (Gamborg). Cell culture media are described in Atlas and Parks (eds) The Handbook of Microbiological Media (1993) CRC Press, Boca Raton, Fla. (Atlas). Additional information is found in commercial literature such as the Life Science Research Cell Culture catalogue (1998) from Sigma-Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS).

In one embodiment of this invention, to confer systemic herbicide tolerance to plants, recombinant DNA vectors which contain isolated sequences and are suitable for transformation of plant cells are prepared. A DNA sequence coding for the desired nucleic acid, for example a cDNA or a genomic sequence encoding a full length protein, is conveniently used to construct a recombinant expression cassette which can be introduced into the desired plant. An expression cassette will typically comprise a selected shuffled nucleic acid sequence operably linked to a promoter sequence and other transcriptional and translational initiation regulatory sequences which will direct the transcription of the sequence from the gene in the intended tissues (e.g., entire plant, leaves, roots) of the transformed plant.

For example, a strongly or weakly constitutive plant promoter can be employed which will direct expression of a shuffled P450 or other enzyme as set forth herein in all tissues of a plant. Such promoters are active under most environmental conditions and states of development or cell differentiation. Examples of constitutive promoters include the 1′- or 2′-promoter derived from T-DNA of Agrobacterium tumefaciens, and other transcription initiation regions from various plant genes known to those of skill. Where overexpression of an herbicide tolerance factor is detrimental to the plant, one of skill, upon review of this disclosure, will recognize that weak constitutive promoters can be used for low-levels of expression. In those cases where high levels of expression is not harmful to the plant, a strong promoter, e.g., a t-RNA or other pol III promoter, or a strong pol II promoter, such as the cauliflower mosaic virus promoter, can be used.

Alternatively, a plant promoter may be under environmental control. Such promoters are referred to here as “inducible” promoters. Examples of environmental conditions that may effect transcription by inducible promoters include pathogen attack, anaerobic conditions, or the presence of light.

In one embodiment of this invention, the promoters used in the constructs of the invention will be “tissue-specific” and are under developmental control such that the desired gene is expressed only in certain tissues, such as leaves and roots.

The endogenous promoters from P450 monooxygenases, glutathione sulfur transferases, homoglutathione sulfur transferases, glyphosate oxidases and 5-enolpyruvylshikimate-3-phosphate synthases are particularly useful for directing expression of these genes to the transfected plant.

Tissue-specific promoters can also be used to direct expression of heterologous structural genes, including shuffled nucleic acids as described herein. Thus, the promoters can be used in recombinant expression cassettes to drive expression of any gene whose expression upon herbicide application is desirable. Examples include genes encoding proteins which ordinarily provide the plant with herbicide tolerance and genes that encode useful phenotypic characteristics, e.g., which influence heterosis.

In general, the particular promoter used in the expression cassette in plants depends on the intended application. Any of a number of promoters which direct transcription in plant cells can be suitable. The promoter can be either constitutive or inducible. In addition to the promoters noted above, promoters of bacterial origin which operate in plants include the octopine synthase promoter, the nopaline synthase promoter and other promoters derived from native Ti plasmids. See, Herrara-Estrella et al. (1983), Nature, 303:209-213. Viral promoters include the 35S and 19S RNA promoters of cauliflower mosaic virus. See, Odell et al. (1985) Nature, 313:810-812. Other plant promoters include the ribulose-1,3-bisphosphate carboxylase small subunit promoter and the phaseolin promoter. The promoter sequence from the E8 gene and other genes may also be used. The isolation and sequence of the E8 promoter is described in detail in Deikman and Fischer, (1988) EMBO J. 7:3315-3327.

To identify candidate promoters, the 5′ portions of a genomic clone is analyzed for sequences characteristic of promoter sequences. For instance, promoter sequence elements include the TATA box consensus sequence (TATAAT), which is usually 20 to 30 base pairs upstream of the transcription start site. In plants, further upstream from the TATA box, at positions -80 to -100, there is typically a promoter element with a series of adenines surrounding the trinucleotide G (or T) N G. Messing et al., Genetic Engineering in Plants, Kosage, et al. (eds.), pp. 221-227 (1983).

In preparing expression vectors of the invention, sequences other than the promoter and the shuffled gene are also preferably used. If proper polypeptide expression is desired, a polyadenylation region at the 3′-end of the shuffled coding region should be included. The polyadenylation region can be derived from the natural gene, from a variety of other plant genes, or from T-DNA. Signal/localization peptides, which e.g., facilitate translocation of the expressed polypeptide to internal organelles (e.g., chloroplasts) or extracellular secretion, may also be employed.

The vector comprising the shuffled sequence will typically comprise a marker gene which confers a selectable phenotype on plant cells. For example, the marker may encode biocide tolerance, particularly antibiotic tolerance, such as tolerance to kanamycin, G418, bleomycin, hygromycin, or herbicide tolerance, such as tolerance to chlorosluforon, or phosphinothricin (the active ingredient in the herbicides bialaphos and Basta—two additional herbicides that, in addition to acting as a selection agent, can be targets of DNA shuffling as set forth hereinabove). Reporter genes, which are used to monitor gene expression and protein localization via visualizable reaction products (e.g., beta-glucoronidase, beta-galactosidase, and chloramphenicol acetyltransferase) or by direct visualization of the gene product itself (e.g., green fluorescent protein (GFP); Sheen et al. (1995) The Plant Journal 8:777-784) may be used for, e.g., monitoring transient gene expression in plant cells. Transient expression systems may be employed in plant cells, for example, in screening plant cell cultures for herbicide tolerance activities.

Plant Transformation

Protoplasts

Numerous protocols for establishment of transformable protoplasts from a variety of plant types and subsequent transformation of the cultured protoplasts are available in the art and are incorporated herein by reference. For examples, see Hashimoto et al. (1990) Plant Physiol. 93: 857; Plant Protoplasts, Fowke L C and Constabel F, eds., CRC Press (1994); Saunders et al. (1993) Applications of Plant In Vitro Technology Symposium, UPM, 16-18 Nov. 1993; and Lyznik et al. (1991) BioTechniques 10: 295, each of which is incorporated herein by reference.

Chloroplasts

Chloroplasts are a proposed site of action of some herbicide tolerance activities, and, in some instances, the herbicide tolerance gene products are preferably fused to chloroplast transit sequence peptides to facilitate translocation of the gene products into the chloroplasts. In these instances, it can be advantageous to transform the shuffled herbicide tolerance nucleic acids into chloroplasts of the plant host cells. Numerous methods are available in the art to accomplish chloroplast transformation and expression (Daniell et al. (1998) Nature Biotechnology 16: 346; O'Neill et al. (1993) The Plant Journal 3: 729; Maliga P (1993) TIBTECH 11: 01). The expression construct comprises a transcriptional regulatory sequence functional in plants operably linked to a polynucleotide encoding the herbicide tolerance gene product. With reference to expression cassettes which are designed to function in chloroplasts (such as an expression cassette comprising a herbicide tolerance nucleic acid encoding a glyphosate tolerant EPSP synthase or a novel EPTD of the present invention), the expression cassette comprises the sequences necessary to ensure expression in chloroplasts. Typically the coding sequence is flanked by two regions of homology to the chloroplastid genome so as to effect a homologous recombination with the genome; often a selectable marker gene is also present within the flanking plastid DNA sequences to facilitate selection of genetically stable transformed chloroplasts in the resultant transplastonic plant cells (see Maliga P (1993) and Daniell et al. (1998), and references cited therein).

General Transformation Methods

DNA constructs of the invention may be introduced into the genome of the desired plant host by a variety of conventional techniques. Techniques for transforming a wide variety of higher plant species are well known and described in the technical and scientific literature. See, e.g., Payne, Gamborg, Atlas, Sigma-LSRCCC and Sigma-PCCS, all supra, as well as, e.g., Weising, et al., (1988) Ann. Rev. Genet. 22:421-477.

For example, DNAs may be introduced directly into the genomic DNA of a plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski, et al., EMBO J. 3:2717-2722 (1984). Electroporation techniques are described in Fromm, et al., Proc. Natl. Acad. Sci. USA 82:5824 (1985). Ballistic transformation techniques are described in Klein, et al., Nature 327:70-73 (1987); and Weeks, et al., Plant Physiol. 102:1077-1084 (1993).

In a particularly preferred embodiment Agrobacterium tumefaciens-mediated transformation techniques are used to transfer shuffled coding sequences to transgenic plants. Agrobacterium-mediated transformation is useful primarily in dicots, however, certain monocots can be transformed by Agrobacterium. For instance, Agrobacterium transformation of rice is described by Hiei, et al., (1994) Plant J. 6:271-282; U.S. Pat. No. 5,187,073; U.S. Pat. No. 5,591,616; Li, et al., (1991) Science in China 34:54; and Raineri, et al., (1990)Bio/Technology 8:33 (1990). Transformed maize, barley, triticale and asparagus by Agrobacterium infection is described in Xu, et al., (1990) Chinese J. Bot. 2:81.

In this technique, the ability of the tumor-inducing (Ti) plasmid of A. tumefaciens to integrate into a plant cell genome is used advantageously to co-transfer a nucleic acid of interest into a recombinant plant cell of this invention. Typically, an expression vector is produced wherein the nucleic acid of interest is ligated into an autonomously replicating plasmid which also contains T-DNA sequences. T-DNA sequences typically flank the expression cassette nucleic acid of interest and comprise the integration sequences of the plasmid. In addition to the expression cassette, T-DNA also typically comprises a marker sequence, e.g., antibiotic tolerance genes. The plasmid with the T-DNA and the expression cassette are then transfected into Agrobacterium tumefaciens. For effective transformation of plant cells, the A. tumefaciens bacterium also comprises the necessary vir regions on a native Ti plasmid.

In an altemative transformation technique, both the T-DNA sequences as well as the vir sequences are on the same plasmid. For a discussion of A. tumefaciens gene transformation, see, Firoozabady & Kuehnle, Plant Cell, Tissue and Organ Culture: Fundamental Methods. Gamborg & Phillips (Eds.), Springer Lab Manual (1995).

For transformation of the plants of this invention in one aspect, explants are made of the tissues of desired plants, e.g., leaves. The explants are then incubated in a solution of A. tumefaciens at about 0.8×10⁹ to about 1.0×10⁹ cells/mL for a suitable time, typically several seconds. The explants are then grown for approximately 2 to 3 days on suitable medium.

Regeneration of Transizenic Plants

Transformed plant cells which are derived by plant transformation techniques, including those discussed above, can be cultured to regenerate a whole plant which possesses the transformed genotype and thus the desired phenotype such as systemic acquired tolerance to an herbicide. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., Protoplasts Isolation and Culture, Handbook of Plant Cell Culture, pp. 124-176, Macmillan Publishing Company, New York, 1983; and Binding, Regeneration of Plants, Plant Protoplasts pp. 21-73, CRC Press, Boca Raton, 1985. Regeneration can also be obtained from plant callus, explants, organs, or parts thereof. Such regeneration techniques are described generally in Klee, et al., Ann. Rev. of Plant Phys. 38:467-486 (1987). See also, Payne, Gamborg, Atlas, Sigma-LSRCCC and Sigma-PCCS, all supra.

After transformation with Agrobacterium, the explants are transferred to selection media. One of skill will realize that the selection media depends on which selectable marker was co-transfected into the explants. After a suitable length of time, transformants will begin to form shoots. After the shoots are about 1 to 2 cm in length, the shoots should be transferred to a suitable root and shoot media. Selection pressure should be maintained once in the root and shoot media.

The transformants will develop roots in 1 to about 2 weeks and form plantlets. After the plantlets are from about 3 to about 5 cm in height, they should be placed in sterile soil in fiber pots. Those of skill in the art will realize that different acclimation procedures should be used to obtain transformed plants of different species. In a preferred embodiment, cuttings, as well as somatic embryos of transformed plants, after developing a root and shoot, are transferred to medium for establishment of plantlets. For a description of selection and regeneration of transformed plants, see, Dodds & Roberts, Experiments in Plant Tissue Culture, 3rd Ed., Cambridge University Press (1995).

The transgenic plants of this invention can be characterized either genotypically or phenotypically to determine the presence of the shuffled gene. Genotypic analysis is the determination of the presence or absence of particular genetic material. Phenotypic analysis is the determination of the presence or absence of a phenotypic trait. A phenotypic trait is a physical characteristic of a plant determined by the genetic material of the plant in concert with environmental factors. The presence of shuffled DNA sequences can be detected as described in the preceding sections on identification of an optimized shuffled nucleic acid, e.g., by PCR amplification of the genomic DNA of a transgenic plant and hybridization of the genomic DNA with specific labeled probes. The survival of plants on a selected herbicide can also be used to monitor incorporation of an herbicide tolerance factor into the plant.

Plants which are transduced with shuffled nucleic acids as taught herein to achieve herbicide tolerance. Essentially any plant can acquire herbicide tolerance by the techniques herein. Some suitable plants for acquisition of herbicide tolerance include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, Malus, Apium, and Datura, including sugarcane, sugar beet, cotton, fruit trees, and legumes. Especially suitable are grass family crops such as maize, wheat, barley, oats, alfalfa, rice, millet, rye and the like. Industrially important legume crops such as soybeans are also especially suitable.

Rapid Evolution as a Predictive Tool

Recursive sequence recombination can be used to simulate natural evolution of plant cells (e.g., weed plant cells) in response to exposure to a herbicide under test. One objective is to identify herbicides for which evolutionary acquisition of tolerance in weeds (or, in a subset of weeds) can be acquired only slowly, if at all. Using whole genome shuffling formats (discussed supra), evolution of plant cells proceeds at a faster rate than in natural evolution. One measure of the rate of evolution is the number of cycles of recombination and screening required until the cells acquire a defined level of tolerance to the herbicide. The information from this analysis is of value in comparing the relative merits of different herbicides and, in particular, in evaluating the long-term efficacy of such herbicides upon repeated administration to weeds.

The plant cells and DNAs used in this analysis may be derived from, e.g., common and/or commercially significant weeds, such as for example, Abutilon threophrasti (velvet leaf), Chenopodium spp. (lambsquarter), Amaranthus spp. (pigweed), Ipomoea spp. (morning glory), Setaria spp. (foxtail), Echinochloa spp., Solanum spp., Sorghum halopense, Digitaria spp., Panicum spp., Bromus tectorum, Kochia scoparia, and the like. Evolution is effected by transforming cells or protoplasts of a plant (such as, one of the weeds described above) that is sensitive to a herbicide under test with a library of DNA fragments, where at least one member of the library is homologous to the native plant genome. The fragments can be, for example, a mutated version of the genome of the plant being evolved. If the target of the herbicide is a known protein or nucleic acid, a focused library containing variants of the corresponding gene can be used. Alternatively, the library can comprise DNA from other kinds of plants, especially weed plants, thereby simulating the source material available for recombination in vivo. The library can also comprise DNA from weeds or other plants known to be tolerant to the herbicide. After transformation and propagation of cells for an appropriate period to allow for recombination to occur and recombinant genes to be expressed, the cells are screened by exposing them to the herbicide under test (at an initial concentration, e.g., which is lethal to 90-95% of the cells) and then collecting survivors. Surviving cells are subject to further rounds of recombination. The subsequent round can be effected by a split and pool approach in which DNA from one subset of surviving cells is introduced into a second subset of cells. Alternatively, a fresh library of DNA fragments can be introduced into surviving cells. Subsequent round(s) of selection can be performed at increasing concentrations of herbicide, thereby increasing the stringency of selection, until resistance to a predetermined level of herbicide has been acquired. The predetermined level of herbicide resistance may reflect the maximum level of a herbicide practical to administer to a crop. The analysis method is valuable for investigating long-term acquisition in weeds of tolerance to various herbicides, such as norflurazon, trifluralin, pendamethalin, sethoxadim, dichlofop-methyl, imazethapyr, dicamba, glufosinate, fomesafen, lactofen, and the like. The method would be especially useful for evaluating the potential for long-term acquisition of tolerance in weeds to newer herbicides, including those with novel modes of action, such as sulcotrione and isoxaflutole. The analysis method is particularly valuable for evaluating long-term acquisition of tolerance to combinations of herbicides.

The value of this analysis can be further enhanced by first applying the method to herbicides for which the facility by which plants acquire tolerance is already known. Examples of herbicides which can be used as standards in the analysis include herbicides which are known to acquire tolerance relatively rapidly in plants, such as chlorsulfuron and atrazine, and herbicides which are known to acquire tolerance relatively slowly in plants, such as glyphosate and metolachlor.

Modifications can be made to the method and materials as hereinbefore described without departing from the spirit or scope of the invention as claimed, and the invention can be put to a number of different uses, including:

The use of an integrated system to test herbicide tolerance in shuffled DNAs, including in an iterative process.

The use of an integrated system to predict long-term efficacy of herbicides in shuffled DNAs, including in an iterative process.

An assay, kit or system utilizing a use of any one of the screening or selection strategies, materials, components, methods or substrates hereinbefore described. Kits will optionally additionally comprise instructions for performing methods or assays, packaging materials, one or more containers which contain assay, device or system components, or the like.

In an additional aspect, the present invention provides kits embodying the methods and apparatus herein. Kits of the invention optionally comprise one or more of the following: (1) a shuffled library as described herein; (2) instructions for practicing the methods described herein, and/or for operating the screening or selection procedures herein; (3) one or more herbicide assay component; (4) a container for holding herbicide, nucleic acid, plant, cell, or the like and, (5) packaging materials.

In a further aspect, the present invention provides for the use of any component or kit herein, for the practice of any method or assay herein, and/or for the use of any apparatus or kit to practice any assay or method herein.

### EXAMPLES

The following examples are offered to illustrate, but not to limit the present invention. Essentially equivalent variations upon the exact procedures set forth will be apparent to one of skill upon review of the present disclosure.

### Example 1

### Shuffling of Plant EPSPS Genes for Glyphosate Tolerance

Arabidopsis EPSPS cDNA is PCR amplified from reverse transcribed RNA using the primers 5′-GCAGT CCATG GAGAA AAGCG TCGGA GATTG TACTT CAACC C-3′ and 5′-TAGAC TAAGA TCTGT GCTTT GTGAT TCTTT CAAGT ACTTG G-3′. Digestion of the fragment with NcoI and BglII is followed by directional cloning into the prokaryotic expression vector pQE60 (QIAGEN) and introduction into the E. coli AroA-strain AB2829 (Pittard, 1966). Likewise, a tomato cDNA is amplified with the primers 5′-ACGTC CATGG CAAAA CCCCA TGAGA TTGTG CTAG-3′ and 5′ CAGTA GATCT GTGCT TAGAG TACTT CTGGA G-3′ from purified phage DNA of a cDNA library (Stratagene), cloned into pQE60, and introduced into AB2829 cells. Growth of the transformed cells on minimal media devoid of aromatic amino acids demonstrates functional complementation of the AroA mutation by expression of the cloned EPSPS genes.

Universal M13 forward and reverse primers are used to PCR amplify both the Arabidopsis and tomato EPSPS genes from the pQE60 clones. The two DNAs are mixed, DNAse treated, and shuffled. The NcoI and BglII primers for Arabidopsis and tomato are mixed and used to amplify shuffled products from the final reassembly mix. The shuffled genes are cloned into pQE60 and electroporated into AB2829 cells. Transformed cells are plated onto minimal media and replica plated onto minimal media plates containing 2, 5, 10 and 20 mM glyphosate. All plates also contain 75 mg/L ampicillin.

Functional, glyphosate-tolerant clones are grown in LB media, induced by IPTG and EPSPS protein purified using a His-Tag purification system (QIAGEN). Activity, and binding kinetics for glyphosate and PEP, are tested using purified enzymes as described in Example 2.

### EXAMPLE 2

### Tolerance to Glyphosate in Recombinant Forms of EPSP Synthase

EPSP synthase activity is assayed in the forward direction by monitoring production of phosphate with the malachite green colorimetric assay (Lanzetta P A et al., Anal. Biochem. 100:95-97, 1979). Reactions are performed in assay buffer (50 mM HEPES, pH 7.0 and 0.1 mM ammonium molybdate) containing enzyme, 0.1 mM phosphoenolpyruvate, 0.1 mM shikimate-3-phosphate and various concentrations of glyphosate, in a final volume of 0.2 ml. After 20 min, reactions are terminated by the addition of 0.7 ml of malachite green reagent (3 parts of 0.045% malachite green to 1 part 4.2% ammonium molybdate). After 10 min, absorbance at 660 nm is determined with a Beckman DU 600 spectrophotometer. The inhibition constant of each enzyme for glyphosate (I50) is derived from a plot of percent activity versus glyphosate concentration. The Kₘ for PEP is derived from a plot of rate of rate of product formed versus PEP concentration.

While the foregoing invention has been described in some detail for purposes of clarity and understanding, it will be clear to one skilled in the art from a reading of this disclosure that various changes in form and detail can be made without departing from the true scope of the invention. For example, all the techniques and materials described above can be used in various combinations. All publications and patent documents cited in this application are incorporated by reference in their entirety for all purposes to the same extent as if each individual publication or patent document were so individually denoted.

1. A method of obtaining a recombinant herbicide tolerance nucleic acid which can confer tolerance to an herbicide upon a plant in which the recombinant herbicide tolerance nucleic acid is present, the method comprising:
(i) recombining a plurality of variant forms of one or more parental nucleic acid, wherein the plurality of variant forms comprises segments derived from the parental nucleic acid, wherein the parental nucleic acid encodes an herbicide tolerance activity, or can be shuffled to encode an herbicide tolerance activity, and wherein the plurality of variant forms differ from each other in at least one nucleotide, to produce a library of recombinant nucleic acids;
(ii) screening the library to identify at least one recombinant herbicide tolerance nucleic acid, wherein the recombinant herbicide tolerance nucleic acid encodes an activity which confers herbicide tolerance to a cell.

2. The method of claim 1, wherein the recombinant herbicide tolerance nucleic acid encodes a distinct or improved herbicide tolerance activity compared to the parental nucleic acid.

3. The method of claim 1, wherein the one or more parental nucleic acid encodes an herbicide tolerance activity.

4. The method of claim 1, wherein the parental nucleic acids do not encode herbicide tolerance activity, wherein recombining the plurality of variant forms provides a nucleic acid which encodes an herbicide tolerance activity.

5. The method of claim 1, wherein the parental nucleic acid encodes a polypeptide which is functionally or structurally similar to a herbicide target protein.

6. The method of claim 1, wherein the plurality of variant forms of the parental nucleic acid comprise allelic or interspecific variants of the parental nucleic acid.

7. The method of claim 1, wherein the plurality of variant forms of the parental nucleic acid is produced by synthesizing a plurality of nucleic acids homologous to the parental nucleic acid.

8. The method of claim 1, wherein the plurality of variant forms of the parental nucleic acid is produced by error-prone transcription of the parental nucleic acid or by replication of the parental nucleic acid in a mutator cell strain.

9. The method of claim 1, wherein the parental nucleic acid encodes a polypeptide or polypeptide fragment selected from the group consisting of: a P450 monooxygenase polypeptide, a glutathione sulfur transferase polypeptide, a homoglutathione sulfur transferase polypeptide, a glyphosate oxidase polypeptide, a phosphinothricin acetyl transferase polypeptide, a dichlorophenoxyacetate monooxygenase polypeptide, an acetolactate synthase polypeptide, a protoporphyrinogen oxidase polypeptide, a 5-enolpyruvylshikimate-3-phosphate synthase polypeptide, and a UDP-N-acetylglucosamine enolpyruvyltransferase polypeptide.

10. The method of claim 9, wherein the parental nucleic acid is selected from the group consisting of: a P450 monooxygenase gene from corn or wheat, a glutathione sulfur transferase gene from corn, a homoglutathione sulfur transferase gene from soybean, a glyphosate oxidase gene from a bacteria, a phosphinothricin acetyl transferase gene from a bacteria, a dichlorophenoxyacetate monooxygenase gene from a bacteria, an acetolactate synthase gene from a plant, a protoporphyrinogen oxidase gene from a plant, a protoporphyrinogen oxidase gene from an alga, an enolpyruvylshikimate-3-phosphate synthase gene from a bacteria, a enolpyruvylshikimate-3-phosphate synthase gene from a plant, and a UDP-N-acetylglucosamine enolpyruvyltransferase gene from a bacteria.

11. The method of claim 5, wherein the parental nucleic acid encodes a UDP-N-acetylglucosamine enolpyruvyltransferase, and wherein the herbicide is glyphosate.

12. The method of claim 1, wherein the library comprises a recombinant nucleic acid produced by recombining a plurality of variant forms of a parental nucleic acid selected from the group consisting of:
a P450 monooxygenase nucleic acid, a homoglutathione sulfur transferase nucleic acid, a glutathione sulfur transferase nucleic acid, a glyphosate oxidase nucleic acid, a phosphinothricin acetyl transferase nucleic acid, a dichlorophenoxyacetate monooxygenase nucleic acid, a acetolactate synthase nucleic acid, a enolpyruvylshikimate-3-phosphate synthase nucleic acid, and a UDP-N-acetylglucosamine enolpyruvyltransferase nucleic acid.

13. The method of claim 1, wherein the screening comprises a step selected from the group consisting of:
(a) screening for oxidation of the herbicide;
(b) screening for glutathione conjugation to the herbicide or to a metabolite of the herbicide;
(c) screening for homoglutathione conjugation to the herbicide or to a metabolite of the herbicide.

14. The method of claim 1, wherein the library of recombinant nucleic acids is present in a population of cells.

15. The method of claim 14, wherein the screening comprises growing the population of cells in or on a medium comprising the herbicide and detecting a physical difference between the herbicide and a modified form of the herbicide produced by the cells.

16. The method of claim 15, wherein the physical difference between the herbicide and the modified form of the herbicide is detected by a difference in fluorescence or absorbance between the herbicide and the modified form of the herbicide.

17. The method of claim 16, wherein the herbicide is dicamba, the recombinant herbicide tolerance nucleic acid encodes a dicamba oxidation activity, and the cells are screened for dicamba oxidation by fluorescence of an oxidized form of dicamba.

18. The method of claim 14, wherein the screening comprises growing the population of cells in or on a medium comprising the herbicide and selecting for enhanced growth of the cells in the presence of the herbicide.

19. The method of claim 18, wherein enhanced growth of the cells requires the activity encoded by the recombinant herbicide tolerance nucleic acid.

20. The method of claim 19, wherein enhanced growth of the cells requires the product of the reaction of the herbicide by the activity encoded by the recombinant herbicide tolerance nucleic acid.

21. The method of claim 20, wherein the cell is an Mpu⁺ strain of bacteria, the herbicide is glyphosate, and the recombinant herbicide tolerance nucleic acid encodes an activity that catalyses the conversion of glyphosate to aminomethylphosphonate.

22. The method of claim 19, wherein the cells are an AroA⁻ strain of bacteria, the herbicide is glyphosate, and the recombinant herbicide tolerance nucleic acid encodes an activity which catalyses the conversion of phosphoenolpyruvate plus shikimate 3-phosphate to 5-enolpyruvylshikimate-3-phosphate.

23. The method of claim 1, the method further comprising screening the library for one or more additional activity that confers tolerance to one or more additional herbicide.

24. The method of claim 1, wherein the step of recombining is performed in a plurality of cells.

25. The method of claim 24, further comprising:
(a) recombining DNA from the plurality of cells that encode herbicide tolerance activity with a second library of DNA fragments, at least one of which undergoes recombination with a segment in a nucleic acid present in the cells to produce recombined cells, or recombining DNA between the plurality of cells that encode herbicide tolerance activity to produce modified cells.

26. The method of claim 25, further comprising:
(b) recombining and screening the recombined or modified cells to produce further recombined cells that have evolved additionally distinct or improved herbicide tolerance activity.

27. The method of claim 26, further comprising:
repeating (a) or (b) until the further recombined cells have acquired additionally distinct or improved herbicide tolerance activity.

28. The method of claim 1, wherein the method further comprises:
(iii) recombining at least one recombinant herbicide tolerance nucleic acid with a further nucleic acid, wherein the further nucleic acid is the same or different from one or more of the plurality of the variant forms of (i), to produce a further library of recombinant nucleic acids;
(iv) screening the further library to identify at least one further recombinant herbicide tolerance nucleic acid that encodes a further improved herbicide tolerance activity compared to a non-recombinant herbicide tolerance gene; and, optionally, repeating (iii) and (iv).

29. The method of claim 28, wherein the further recombinant herbicide tolerance nucleic acid encodes two or more distinct or improved herbicide tolerance activities.

30. The method of claim 1, wherein the library is present in bacterial cells and the method comprises:
pooling multiple separate library members;
screening the resulting pooled library members for a recombinant herbicide tolerance nucleic acid that encodes a distinct or improved herbicide tolerance activity compared to a non-recombinant herbicide tolerance nucleic acid; and,
cloning the distinct or improved recombinant herbicide tolerance nucleic acid.

31. The method of claim 2, wherein the distinct or improved herbicide tolerance activity is selected from the group consisting of: an increase in ability to metabolize the herbicide; an increase in the range of herbicides to which the activity confers tolerance; an increase in expression level compared to that of a polypeptide encoded by the parental nucleic acid; a decrease in susceptibility to inhibition by the herbicide compared to that of an activity encoded by the parental nucleic acid; a decrease in susceptibility to protease cleavage compared to that of a polypeptide encoded by the parental nucleic acid; a decrease in susceptibility to high or low pH levels compared to that of a polypeptide encoded by the parental nucleic acid; a decrease in susceptibility to high or low temperatures compared to that of a polypeptide encoded by the parental nucleic acid; a decrease in toxicity to a host plant compared to that of a polypeptide encoded by the selected nucleic acid; and any combination of two or more thereof.

32. The method of claim 1, further comprising transducing the recombinant herbicide tolerance nucleic acid into a plant.

33. The method of claim 1, further comprising transducing the recombinant herbicide tolerance nucleic acid into a plant and testing the resulting transduced plant for tolerance to the herbicide.

34. The method of claim 1, further comprising transducing the recombinant herbicide tolerance nucleic acid into a plant and breeding the plant with a separate plant strain of the same species, followed by selection of resulting offspring for tolerance to the herbicide.

35. A library of recombinant nucleic acids made by the method of claim 1.

36. The library of claim 35, wherein the library is a phage display library.

37. A recombinant herbicide tolerance nucleic acid made by the method of claim 1.

38. A DNA shuffling mixture comprising at least three homologous DNAs, wherein each of the at least three homologous DNAs is derived from a parental nucleic acid encoding a polypeptide or polypeptide fragment selected from the group consisting of: a P450 monooxygenase, a glutathione sulfur transferase, a homoglutathione sulfur transferase, a glyphosate oxidase, a phosphinothricin acetyl transferase, a dichlorophenoxyacetate monooxygenase, an acetolactate synthase, a protoporphyrinogen oxidase, a 5-enolpyruvylshikimate-3-phosphate synthase, and a UDP-N-acetylglucosamine enolpyruvyltransferase.

39. The DNA shuffling mixture of claim 38, wherein the at least three homologous DNAs are present in cell culture, in vitro, or in a plant.

40. The DNA shuffling mixture of claim 38, wherein the homologous DNAs are derived from a parental nucleic acid encoding a P450 monooxygenase from corn or wheat.

41. The DNA shuffling mixture of claim 38, wherein at least one of the homologous DNAs is derived from a parental nucleic acid encoding a glutathione sulfur transferase from maize.

42. The DNA shuffling mixture of claim 38, wherein at least one of the homologous DNAs is derived from a parental nucleic acid encoding a homoglutathione sulfur transferase from soybean.

43. The DNA shuffling mixture of claim 38, wherein at least one of the homologous DNAs is derived from a parental nucleic acid encoding a glyphosate oxidase from a bacteria.

44. The DNA shuffling mixture of claim 38, wherein at least one of the homologous DNAs is derived from a parental nucleic acid encoding a phosphinothricin acetyl transferase from a bacteria.

45. The DNA shuffling mixture of claim 38, wherein at least one of the homologous DNAs is derived from a parental nucleic acid encoding a dichlorophenoxyacetate monooxygenase from a bacteria.

46. The DNA shuffling mixture of claim 38, wherein at least one of the homologous DNAs is derived from a parental nucleic acid encoding an acetolactate synthase from a plant.

47. The DNA shuffling mixture of claim 38, wherein at least one of the homologous DNAs is derived from a parental nucleic acid encoding a 5-enolpyruvylshikimate-3-phosphate synthase from a bacteria.

48. The DNA shuffling mixture of claim 38, wherein at least one of the homologous DNAs is derived from a parental nucleic acid encoding a 5-enolpyruvylshikimate-3-phosphate synthase from a plant.

49. The DNA shuffling mixture of claim 38, wherein at least one of the homologous DNAs is derived from a parental nucleic acid encoding a UDP-N-acetylglucosamine enolpyruvyltransferase from a bacteria.

50. The DNA shuffling mixture of claim 38, wherein at least one of the homologous DNAs is derived from a parental nucleic acid encoding a protoporphyrinogen oxidase from a plant or an alga.

51. A method of acquiring or improving an herbicide tolerance activity in a parental plant cell, comprising performing whole genome shuffling of a plurality of genomic nucleic acids in the plant cell, forming a modified plant cell, and screening the modified plant cell for a distinct or improved tolerance activity to one or more herbicide compared to the parental plant cell.

52. The method of claim 51, wherein the herbicide is selected from the group consisting of: dicamba, glyphosate, bisphosphonate, sulfentrazone, imidazolinone, sulfonylurea, triazolopyrimidine, phenoxyacetic acid, diphenyl ether, chloroacetamide, and hydantocidin.

53. The method of claim 51, wherein the genomic nucleic acids are from a species or strain different from the parental plant cell.

54. The method of claim 51, further comprising: regenerating the modified plant cell, or a descendent cell thereof, into a plant.

55. A method of predicting long-term efficacy of a herbicide in killing a plant, the method comprising:
(i.) transforming a plurality of cells of the plant with a library of DNA fragments at least some of which undergo recombination with segments in the genome of the cells to produce modified plant cells;
(ii.) propagating the modified plant cells in a media containing the herbicide, and recovering surviving plant cells;
(iii.) recombining DNA from surviving plant cells with a further library of DNA fragments at least some of which undergo recombination with cognate segments in the DNA from the surviving plant cells to produce further modified plant cells;
(iv.) propagating further modified plant cells, in media containing the herbicide, and collecting further surviving plant cells;
(v.) repeating (iii.) and (iv.), as necessary, until a further surviving plant cell has acquired a desired degree of resistance to the herbicide, whereby the degree of resistance acquired and the number of repetitions of (iii.) and (iv.) needed to acquire it provide a measure of the long-term efficacy of the herbicide in killing the plant.

56. The method of claim 55, wherein the plant is a weed plant.

57. The method of claim 56, wherein the plant is selected from the group consisting of Abutilon threophrasti, Chenopodium spp., Amaranthus spp., Ipomoea spp., Setaria spp., Echinochloa spp., Solanum spp., Sorghum halopense, Digitaria spp., Panicum spp., Bromus tectorum, and Kochia scoparia.

58. The method of claim 55, fuirther comprising repeatedly recombining DNA from the modified plant cells, wherein the repeated recombination is performed prior to propagating the modified plant cells in a media containing the herbicide.

59. The method of claim 55, further comprising dividing surviving plant cells into first and second pools, isolating the further library of DNA from the first pool and transforming the second pool with the further library.

60. The method of claim 55, wherein the further library of DNA is obtained from a species or strain different from the plant cell.
